(19) United States
(12) Patent Application Publication
(10) Pub. No.: US 2026/0007373 A1
(43) Pub. Date: Jan. 8, 2026

(54) X-RAY DIAGNOSTIC APPARATUS AND MEDICAL BED DEVICE

(71) Applicant: CANON MEDICAL SYSTEMS CORPORATION, Otawara-shi (JP)

(72) Inventor: Tasuku MIYAUCHI, Utsunomiya (JP)

(73) Assignee: CANON MEDICAL SYSTEMS CORPORATION, Otawara-shi (JP)

(21) Appl. No.: 19/249,208

(22) Filed: Jun. 25, 2025

(30) Foreign Application Priority Data
Jul. 5, 2024 (JP) 2024-108887
Jun. 3, 2025 (JP) 2025-092487

(51) Int. Cl. A61B 6/04; A61B 6/00; A61B 6/46

(52) U.S. Cl. CPC A61B 6/0407; A61B 6/467; A61B 6/56

(57) ABSTRACT

An X-ray diagnostic apparatus according to the embodiment comprises a restrictor, a manipulator, and a releaser. The restrictor sets a movement restriction of a top plate in a horizontal direction, wherein a subject is placed on the top plate. The manipulator receives a releasing operation for releasing the movement restriction. The releaser releases the movement restriction when the manipulator receives a first operation that is the releasing operation from below.

### CROSS REFERENCE TO RELATED APPLICATIONS

This application is based upon and claims the benefit of priority from the prior Japanese Patent Applications No. 2024-108887, filed on Jul. 5, 2024, and No. 2025-092487, filed on Jun. 3, 2025, the entire contents of which are incorporated herein by reference.

### FIELD

Embodiments disclosed in the present specification and the drawings relate to an X-ray diagnostic apparatus and a medical bed apparatus.

### BACKGROUND

Conventionally, the couch of the X-ray diagnostic apparatus is provided with a top plate longitudinal/lateral brake release switch (panning knob) for releasing the movement restriction of the top plate in the horizontal direction to enable the top plate to move in the horizontal direction. The operator of the top plate can move the top plate by sliding the top plate in the horizontal direction while pressing down the panning knob. The conventional panning knob is, for example, a simple push-button whose switch cap part has a large mushroom-shaped shape.

During a procedure or the like, since the panning knob is large, the belly of a worker such as a doctor, a nurse, or a radiologist hits the panning knob, which causes an erroneous operation. In addition, dropping an object on the panning knob or touching of a leg or a hand of the subject on the top plate also causes erroneous operation. An erroneous operation by such an object and a person may hinder the progress of the procedure. Therefore, there is a demand for a switch having a structure in which the switch does not operate even if a belly of an operator or the like hits the switch. The above problem is not limited to the couch of the X-ray diagnostic apparatus, and similarly occurs in a couch used together with another apparatus such as MRI or a couch used alone.

### BRIEF DESCRIPTION OF THE DRAWINGS

FIG. 1 is a block diagram illustrating a configuration example of an X-ray diagnostic apparatus according to a first embodiment;

FIG. 2 is a perspective view illustrating an example of a top plate and an operation panel according to the first embodiment;

FIG. 3 is a perspective view illustrating an example of the operation panel and a panning knob according to the first embodiment;

FIG. 4A is a flowchart illustrating an example of a releasing operation in the X-ray diagnostic apparatus according to the first embodiment;

FIG. 4B is a flowchart illustrating another example of the releasing operation in the X-ray diagnostic apparatus according to the first embodiment;

FIG. 5 is a partial cross-sectional view illustrating an example of the releasing operation in the X-ray diagnostic apparatus according to the first embodiment;

FIG. 6 is a partial cross-sectional view illustrating the example of the releasing operation in the X-ray diagnostic apparatus according to the first embodiment subsequent to FIG. 5;

FIG. 7 is a partial cross-sectional view illustrating the example of the releasing operation in the X-ray diagnostic apparatus according to the first embodiment subsequent to FIG. 6;

FIG. 8 is a partial cross-sectional view illustrating an example of an operation panel and a panning knob according to a second embodiment;

FIG. 9 is a flowchart illustrating an example of a releasing operation in the X-ray diagnostic apparatus according to the second embodiment;

FIG. 10 is a partial cross-sectional view illustrating an example of an operation panel and a panning knob according to a first modification of the second embodiment;

FIG. 11 is a partial cross-sectional view illustrating an example of an operation panel and a panning knob according to a second modification of the second embodiment;

FIG. 12 is a partial cross-sectional view illustrating an example of an operation panel and a panning knob according to a third modification of the second embodiment;

FIG. 13 is a partial cross-sectional view illustrating an example of an operation panel and a panning knob according to a third embodiment;

FIG. 14 is a flowchart illustrating an example of a releasing operation in the X-ray diagnostic apparatus according to the third embodiment;

FIG. 15 is a partial cross-sectional view illustrating an example of an operation panel and a panning knob according to a fourth embodiment;

FIG. 16 is a flowchart illustrating an example of a releasing operation in the X-ray diagnostic apparatus according to the fourth embodiment;

FIG. 17 is a perspective view illustrating an example of an enlarged portion of a second detection unit of a panning knob according to a fifth embodiment;

FIG. 18 is a plan view illustrating an example of an enlarged portion of a second detection unit and a first detection unit of a panning knob according to the fifth embodiment;

FIG. 19 is a perspective view illustrating another example of the panning knob according to the fifth embodiment;

FIG. 20 is a flowchart illustrating an example of a releasing operation in the X-ray diagnostic apparatus according to the fifth embodiment;

FIG. 21 is a plan view illustrating an example of the releasing operation in the X-ray diagnostic apparatus according to the fifth embodiment;

FIG. 22 is a partial cross-sectional view illustrating an example of an operation panel and a panning knob according to a sixth embodiment;

FIG. 23 is a flowchart illustrating an example of a releasing operation in the X-ray diagnostic apparatus according to the sixth embodiment;

FIG. 24 is a partial cross-sectional view illustrating an example of the releasing operation in the X-ray diagnostic apparatus according to the sixth embodiment;

FIG. 25 is a partial cross-sectional view illustrating an example of an operation panel and a panning knob according to a seventh embodiment;

FIG. 26 is a perspective view illustrating an example of a top plate and an operation panel according to the seventh embodiment;

FIG. 27 is a flowchart illustrating an example of a releasing operation in the X-ray diagnostic apparatus according to the seventh embodiment;

FIG. 28 is a partial cross-sectional view illustrating an example of an operation panel and a panning knob according to an eighth embodiment;

FIG. 29 is a perspective view illustrating an example of a top plate and an operation panel according to the eighth embodiment;

FIG. 30 is a flowchart illustrating an example of a releasing operation in the X-ray diagnostic apparatus according to the eighth embodiment;

FIG. 31 is a block diagram illustrating a configuration example of an X-ray diagnostic apparatus according to a ninth embodiment;

FIG. 32 is a perspective view illustrating an example of a top plate and an operation panel according to the ninth embodiment;

FIG. 33 is a block diagram illustrating a configuration example of an X-ray diagnostic apparatus according to a tenth embodiment;

FIG. 34 is a block diagram illustrating a configuration example of an X-ray diagnostic apparatus according to an eleventh embodiment;

FIG. 35 is a flowchart illustrating an example of a releasing operation in the X-ray diagnostic apparatus according to the eleventh embodiment;

FIG. 36 is a flowchart illustrating an example of a releasing operation in the X-ray diagnostic apparatus according to a modification of the eleventh embodiment;

FIG. 37 is a block diagram illustrating a configuration example of an X-ray diagnostic apparatus according to a twelfth embodiment;

FIG. 38 is a flowchart illustrating an example of a releasing operation in the X-ray diagnostic apparatus according to the twelfth embodiment; and

FIG. 39 is a flowchart illustrating an example of a releasing operation in the X-ray diagnostic apparatus according to a modification of the twelfth embodiment.

### DETAILED DESCRIPTION

An X-ray diagnostic apparatus according to the embodiment comprises a restrictor, a manipulator, and a releaser. The restrictor sets a movement restriction of a top plate in a horizontal direction, wherein a subject is placed on the top plate. The manipulator receives a releasing operation for releasing the movement restriction. The releaser releases the movement restriction when the manipulator receives a first operation that is the releasing operation from below.

Hereinbelow, embodiments of an X-ray diagnostic apparatus are described with reference to the drawings. Note that in the following description, components having substantially the same functions and configurations are denoted by the same reference numerals, and a repeated description is made only when necessary.

### First Embodiment

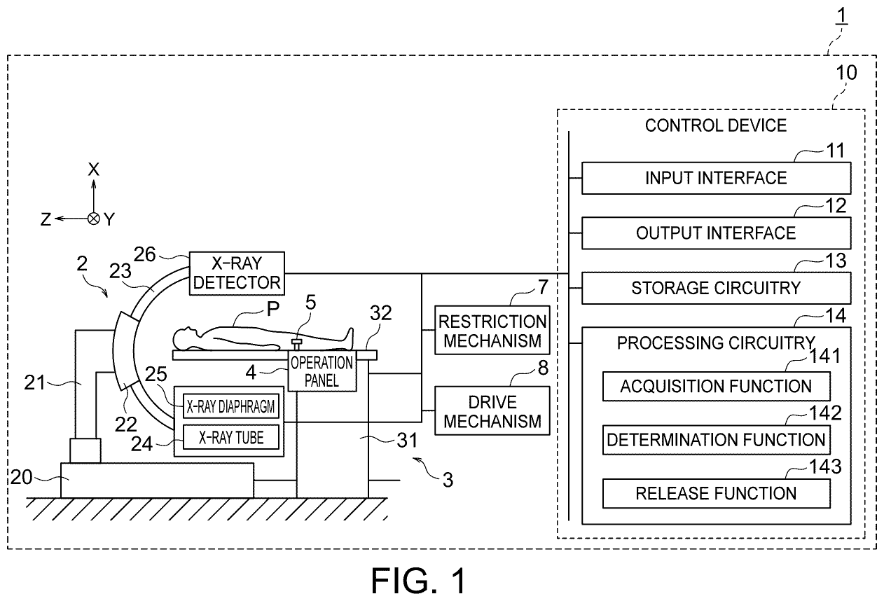

FIG. 1 is a block diagram illustrating a configuration example of an X-ray diagnostic apparatus 1 according to a first embodiment. In the following description, as illustrated in FIG. 1, a horizontal direction along the longitudinal direction of a top plate 32 of a couch 3 is defined as a Z-axis direction, a vertical direction is defined as an X-axis direction, and a direction orthogonal to the X-axis direction and the Z-axis direction is defined as a Y-axis direction.

The X-ray diagnostic apparatus 1 of the present embodiment is an X-ray circulator diagnostic apparatus. The X-ray circulator diagnostic apparatus is also called an X-ray angiography apparatus. Note that the X-ray diagnostic apparatus 1 is not limited to the X-ray circulator diagnostic apparatus, and may be a general X-ray diagnostic apparatus or an X-ray TV apparatus.

As illustrated in FIG. 1, the X-ray diagnostic apparatus 1 according to the present embodiment includes an imaging device 2, a couch 3, an operation panel 4, a panning knob 5, a restriction mechanism 7, a drive mechanism 8, and a control device 10. Among the components of the X-ray diagnostic apparatus 1, for example, the couch 3, the operation panel 4, the panning knob 5, the restriction mechanism 7, the drive mechanism 8, and the control device 10 constitute a medical bed apparatus.

The imaging device 2 is a device for X-ray imaging a subject P. As illustrated in FIG. 1, the imaging device 2 includes a base 20, a stand 21, an arm holder 22, an arm 23, an X-ray tube 24, an X-ray diaphragm 25, and an X-ray detector 26.

The base 20 is provided on a floor surface of an imaging room or the like, and one end portion thereof is rotatably provided with respect to the floor surface. In the example of FIG. 1, the base 20 is provided to pass through a right end portion of the base 20 and to be rotatable with respect to a rotation axis along the X axis. Further, the base 20 rotatably supports the stand 21 on a rotation axis passing through the other end portion of the base 20 and extends along the X-axis. The rotation of the base 20 with respect to the floor surface and the rotation of the stand 21 supported by the base 20 are driven by the drive mechanism 8.

The stand 21 extends upward from the floor surface, and vertically movably supports the arm holder 22 on the upper portion of the stand 21. In addition, the stand 21 rotatably supports the arm holder 22 on a rotation axis passing through the arm holder 22 and along the Z axis. The vertical movement and rotation of the arm holder 22 supported by the stand 21 are driven by the drive mechanism 8.

The arm holder 22 slidably supports the arm 23. The sliding of the arm 23 supported by the arm holder 22 is driven by the drive mechanism 8.

The arm 23 supports the X-ray tube 24 and the X-ray diaphragm 25 at one end, and supports the X-ray detector 26 at the other end.

The X-ray tube 24 is a vacuum tube that generates X-rays by applying a high voltage from an X-ray high voltage device (not illustrated) and supplying a filament current to irradiate thermal electrons from a cathode (filament) toward an anode (target). The thermal electrons collide with the target to generate X-rays. The X-ray tube 24 is, for example, a rotating anode type X-ray tube that generates an X-ray by irradiating a rotating anode with thermal electrons. The type of the X-ray tube 24 is not limited to the rotary anode type, and any type of X-ray tube can be applied.

The X-ray high voltage device includes an electric circuitry such as a transformer and a rectifier, a high-voltage generator, and an X-ray controller. The high-voltage generator has a function of generating a high voltage to be applied to the X-ray tube 24 and a filament current to be supplied to the X-ray tube 24. The X-ray control device controls an output voltage according to the X-ray emitted from the X-ray tube 24. The high-voltage generator may be a transformer type or an inverter type. The X-ray high voltage device may be provided in the imaging device 2.

The X-ray diaphragm 25 is provided on the front surface of the X-ray emission window in the X-ray tube 24. The X-ray diaphragm 25 has, for example, four diaphragm blades made of a metal plate such as lead. The diaphragm blade is driven by the drive mechanism 8 according to the region of interest input by the operator via an input interface 11. The X-ray diaphragm 25 adjusts the region where the X-ray is shielded to any size by sliding the diaphragm blade by the drive mechanism 8. The X-ray diaphragm 25 shields X-rays outside the opening region by the adjusted diaphragm blade. As a result, the X-ray diaphragm 25 narrows down the X-ray generated by the X-ray tube 24 so that the region of interest of the subject P is irradiated with the X-ray.

The X-ray detector 26 detects the X-ray generated by the X-ray tube 24 and transmitted through the subject P. The X-ray detector 26 is, for example, an X-ray flat panel detector (hereinafter referred to as FPD). The FPD includes, for example, a plurality of semiconductor detection elements. The semiconductor detection element includes a direct conversion type in which an X-ray is directly converted into an electric signal and an indirect conversion type in which an X-ray is converted into light by a phosphor and the light is converted into an electric signal. Any type may be used for the FPD. Electric signals generated by the plurality of semiconductor detection elements in association with incidence of X-rays are output to an analog to digital converter (hereinafter referred to as A/D converter) not illustrated. The A/D converter converts the electric signal into digital data. The A/D converter outputs the digital data to the processing circuitry 14. An image intensifier may be used as the X-ray detector 26.

In the example of FIG. 1, the X-ray diagnostic apparatus 1 includes the floor-mounted imaging device 2. The X-ray diagnostic apparatus 1 is not limited to this, and may include a ceiling-suspended imaging device instead of the imaging device 2 or in addition to the imaging device 2.

The couch 3 includes a base 31 and a top plate 32. The base 31 supports the top plate 32 so as to be vertically movable and horizontally movable. The vertical movement and the horizontal movement of the top plate 32 are also called panning. In the present embodiment, the vertical movement of the top plate 32 supported by the base 31 is driven by the drive mechanism 8. On the other hand, the horizontal movement of the top plate 32 supported by the base 31 is performed manually, that is, by hand.

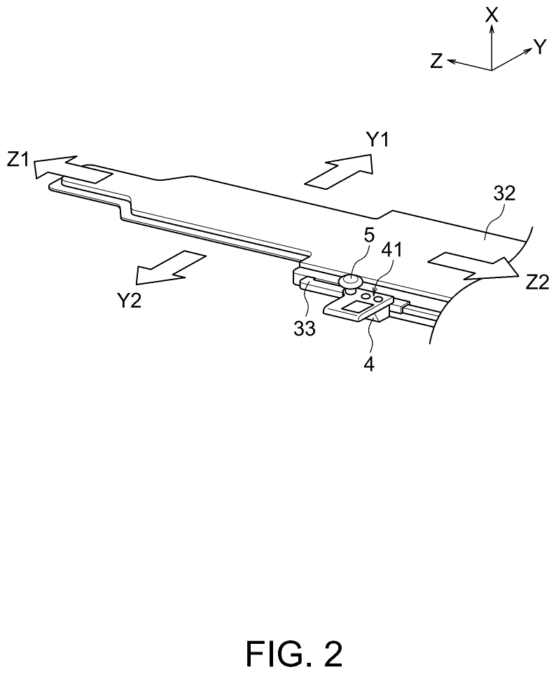

The top plate 32 is a flat plate-like member on which the subject P is placed. FIG. 2 is a perspective view illustrating an example of the top plate 32 and the operation panel 4 according to the first embodiment. As illustrated in FIG. 2, the top plate 32 of the present embodiment has a shape in which the length in the Y-axis direction gradually decreases toward the direction Z1. The shape of the top plate 32 is not limited to the example of FIG. 2 and may be any shape. As described above, the top plate 32 is movable in the vertical direction, that is, in the X-axis direction. The top plate 32 is movable in the horizontal direction with respect to the base 31. That is, as illustrated in FIG. 2, the top plate 32 is movable in a direction Y1 and a direction Y2 along the Y-axis direction, a direction Z1 and a direction Z2 along the Z-axis direction, and any direction on the YZ plane. The top plate 32 may be rotatable about a rotation axis along the X-axis direction, the Y-axis direction, and the Z-axis direction. In the present embodiment, a rail 33 is provided at an edge portion of the top plate 32.

The operation panel 4 is a console device for an operator such as a doctor, a technician, or a nurse to operate the top plate 32. As illustrated in FIG. 2, the operation panel 4 is provided on the rail 33 of the top plate 32. The operation panel 4 includes, for example, an operation button 41 for operating the top plate 32. The operator operates the operation button 41 to adjust the vertical position and the like of the top plate 32. In addition to the operation panel 4, an operation panel for operating the arm 23, the X-ray diaphragm 25, and the like of the imaging device 2, an operation panel for operating a ceiling-suspended imaging device, and the like may be provided on the rail 33. In the present embodiment, the operation panel including the operation panel 4 is provided with a mechanism that hooks onto the rail 33, and these operation panels can be easily attached to or detached from the rail 33. The operator can change the order of these operation panels according to his/her preference and arrange the operation panels at intervals.

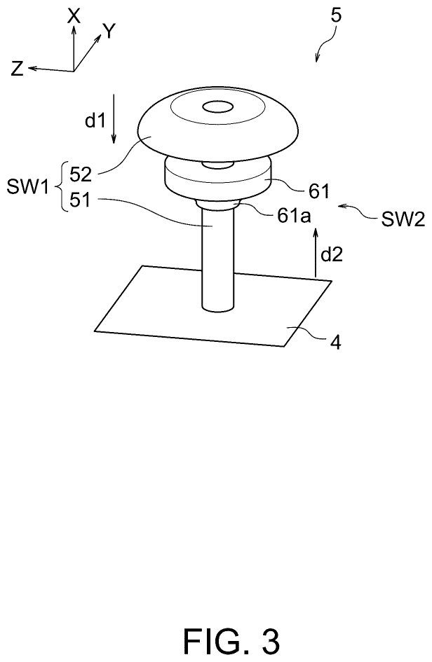

The panning knob 5 is provided on the operation panel 4. The panning knob 5 receives a releasing operation for releasing the movement restriction in the horizontal direction with respect to the top plate 32 on which the subject P is placed. In the present embodiment, the panning knob 5 receives, as such a releasing operation, the first operation that is a releasing operation from below and a second operation that is a releasing operation from above. The panning knob 5 is also referred to as a top longitudinal/lateral brake release switch. The panning knob 5 is an example of a manipulator in the present embodiment. Hereinafter, the panning knob 5 according to the present embodiment will be described in detail with reference to FIG. 3.

FIG. 3 is a perspective view illustrating an example of the operation panel 4 and the panning knob 5 according to the first embodiment. As illustrated in FIG. 3, the panning knob 5 according to the present embodiment includes a switch SW1 and a switch SW2. In the present embodiment, the panning knob 5 receives a second operation, which is a releasing operation from above, by the switch SW1. Also, the panning knob 5 receives the first operation, which is a releasing operation from below, by the switch SW2.

More specifically, the switch SW1 is provided on the panning knob 5 and detects an input from above. The switch SW1 is an example of a second detection unit in the present embodiment. The input from above detected by the switch SW1 is an example of a second input in the present embodiment. The operation to the switch SW1 from above is an example of the second operation in the present embodiment.

In the present embodiment, the switch SW1 is a physical switch. More specifically, as illustrated in FIG. 3, the panning knob 5 includes, as the switch SW1, a support column 51 extending upward from the operation panel 4, and a disk-shaped enlarged portion 52 provided on the support column 51 and enlarged laterally from the support column 51, in which the enlarged portion 52 is moved downward, that is, in a direction d1 illustrated in FIG. 3 to detect an input. That is, in the present embodiment, the input from above detected by the switch SW1 is an input from above to the enlarged portion 52. In other words, the panning knob 5 receives the second operation from above with respect to the panning knob 5 when the enlarged portion 52 is moved downward. For example, the operator presses the palm of one of the right hand and the left hand against the enlarged portion 52 from above to press the enlarged portion 52. When the enlarged portion 52 is moved downward, the switch SW1 is turned on. More specifically, the switch SW1 includes, for example, a detection mechanism provided between the lower portion of the support column 51 and the operation panel 4. In this case, for example, the enlarged portion 52 is immovably fixed to the support column 51, and when the operator presses the enlarged portion 52, the support column 51 moves downward with respect to the operation panel 4 together with the enlarged portion 52. As a result, the detection mechanism of the switch SW1 is turned on. The present invention is not limited thereto, and a detection mechanism of the switch SW1 may be provided between the support column 51 and the enlarged portion 52. In this case, for example, the support column 51 is immovably fixed to the operation panel 4, and when the operator presses the enlarged portion 52, the enlarged portion 52 moves downward with respect to the support column 51. As a result, the detection mechanism of the switch SW1 is turned on.

As described above, in the switch SW1 according to the present embodiment, the enlarged portion 52 has a large mushroom shape. As illustrated in FIG. 3, the upper edge portion of the enlarged portion 52 in the present embodiment is provided with an inclined surface such that the thickness of the enlarged portion 52 decreases from the support column 51 toward the outer periphery. As a result, the operator can easily grip the enlarged portion 52.

On the other hand, the switch SW2 is provided on the panning knob 5 and detects an input from below. The switch SW2 is an example of a first detection unit in the present embodiment. The input from below detected by the switch SW2 is an example of a first input in the present embodiment. The operation to the switch SW2 from below is an example of the first operation in the present embodiment.

The switch SW2 of the present embodiment is a physical switch different from the switch SW1. More specifically, as illustrated in FIG. 3, the panning knob 5 includes, as the switch SW2, a sliding portion 61 provided so as to be slidable on the support column 51 of the switch SW1, and detects an input when the sliding portion 61 is moved upward, that is, in a direction d2 illustrated in FIG. 3. That is, in the present embodiment, the input from below detected by the switch SW2 is an input from below to the sliding portion 61. In other words, the panning knob 5 receives the first operation from below with respect to the panning knob 5 when the sliding portion 61 is moved upward. For example, the operator grips the sliding portion 61 together with the enlarged portion 52 with a hand pressing the enlarged portion 52. As a result, the sliding portion 61 is moved in the upward direction, and the switch SW2 is turned on. More specifically, the switch SW2 includes, for example, a detection mechanism provided on the upper surface of the sliding portion 61. In this case, the detection mechanism of the switch SW2 is turned on by the upward movement of the sliding portion 61.

In the present embodiment, the physical switches constituting the switch SW1 and the switch SW2 are trigger switches. That is, when the operator releases his/her hand from the switch SW1 moved downward, the switch SW1 returns to the upward direction and enters the OFF state. Similarly, when the operator releases his/her hand from the switch SW2 moved upward, the switch SW2 returns to the downward direction and enters the OFF state.

In the present embodiment, the inputs detected by the switch SW1 and the switch SW2 are electronically processed. That is, when the switch SW1 is in the ON state, the switch SW1 outputs an electric signal indicating that the switch SW1 is in the ON state to the control device 10. On the other hand, when the switch SW1 is in the OFF state, the switch SW1 does not output an electric signal. Similarly, when the switch SW2 is in the ON state, the switch SW2 outputs an electric signal indicating that the switch SW2 is in the ON state to the control device 10. On the other hand, when the switch SW2 is in the OFF state, the switch SW2 does not output an electric signal. When the switch SW1 and the switch SW2 are in the OFF state, the switch SW1 and the switch SW2 may output electric signals indicating the OFF state.

As illustrated in FIG. 3, in the present embodiment, the lower edge portion of the sliding portion 61 of the switch SW2 in the panning knob 5 is provided with an inclined surface 61a such that the thickness of the sliding portion 61 decreases from the support column 51 toward the outer periphery. In the present embodiment, the inclined surface 61a has a curved shape recessed toward the support column 51. The present invention is not limited thereto, and the inclined surface 61a may have a linear shape connecting the side surface and the lower surface of the sliding portion 61.

As illustrated in FIG. 1, the restriction mechanism 7 sets the movement restriction in the horizontal direction of the top plate 32 on which the subject P is placed. The restriction mechanism 7 includes, for example, an electromagnetic lock. The electromagnetic lock of the restriction mechanism 7 restricts the movement of the top plate 32 by pressing a component against another portion or hooking the component. The restriction mechanism 7 is not limited to the electromagnetic lock, and may restrict the movement of the top plate 32 in the horizontal direction by another mechanism such as a mechanical mechanism. The restriction mechanism 7 is an example of a restrictor in the present embodiment.

The drive mechanism 8 generates a driving force for performing various movements related to the base 20, the stand 21, the arm holder 22, the arm 23, and the X-ray diaphragm 25 of the imaging device 2 and a movement in the vertical direction related to the top plate 32 of the couch 3. The drive mechanism 8 includes, for example, a motor, an actuator, a gear, and a conveying belt. The drive mechanism 8 is an example of a driver in the present embodiment.

The control device 10 includes an input interface 11, an output interface 12, a storage circuitry 13, and a processing circuitry 14.

The input interface 11 receives various instructions and information input operations from the operator. Specifically, the input interface 11 converts an input operation accepted from the operator into an electric signal, and outputs the electric signal to the processing circuitry 14. For example, the input interface 11 is obtained by using a trackball, a switch button, a mouse, a keyboard, a touch pad that performs an input operation by touching on an operating surface, a touch screen in which a display screen and a touch pad are integrated, a non-contact input circuitry using an optical sensor, a sound input circuitry, etc. The operation panel 4 described above and other operation panels are also included in the input interface 11. Note that the input interface 11 is not limited to one including physical operating components such as a mouse or a keyboard. For example, also an electric signal processing circuitry that receives an electric signal corresponding to an input operation from an external input device provided separately from the apparatus and outputs the electric signal to the control circuitry is included in examples of the input interface 11.

The output interface 12 outputs various types of information. For example, the output interface 12 includes a display. The display converts data of information and images sent from the processing circuitry 14 into an electric signal for displaying, and outputs the electric signal. The display is obtained by using a liquid crystal monitor, a CRT (cathode ray tube) monitor, a touch panel, or the like. The output interface 12 may include a speaker.

The storage circuitry 13 is a non-transitory storage device that stores various types of information, and is, for example, a hard disk drive (HDD), an optical disc, a solid state drive (SSD), an integrated circuit storage device, or the like. The storage circuitry 13 stores, for example, a control program for controlling the X-ray diagnostic apparatus 1 and various pieces of data used to execute the control program. The storage circuitry 13 may be a drive device that reads and writes various types of information from and to a portable storage medium such as a compact disc (CD), a digital versatile disc (DVD), or a flash memory, a semiconductor memory device such as a random-access memory (RAM), or the like, in addition to the HDD, the SSD, and the like.

The processing circuitry 14 is a circuitry that controls the entire operation of the X-ray diagnostic apparatus 1 according to an electric signal of an input operation inputted from the input interface 11. In the present embodiment, the processing circuitry 14 includes an acquisition function 141, a determination function 142, and a release function 143.

Here, for example, the processing functions executed by the acquisition function 141, the determination function 142, and the release function 143, which are components of the processing circuitry 14 illustrated in FIG. 1, are recorded in the storage circuitry 13 in the form of a program executable by a computer. The processing circuitry 14 is, for example, a processor. The processor constituting the processing circuitry 14 reads each program from the storage circuitry 13 and executes the program to implement the function corresponding to the read program. In other words, the processing circuitry 14 that has read each program has the function illustrated in the processing circuitry 14 of FIG. 1.

Note that FIG. 1 illustrates a case where the acquisition function 141, the determination function 142, and the release function 143 are implemented by a single processing circuitry 14, but the embodiment is not limited thereto. For example, the processing circuitry 14 may be configured by combining a plurality of independent processors together, and each processor may achieve one of the acquisition function 141, the determination function 142, and the release function 143 by executing the corresponding program. In addition, each processing function of the processing circuitry 14 may be achieved while being appropriately distributed or integrated into one or a plurality of processing circuitries.

The acquisition function 141 acquires that the switch SW1 and the switch SW2 have detected an input. More specifically, the acquisition function 141 acquires an electric signal indicating that the switch SW1 is in the ON state and an electric signal indicating that the switch SW2 is in the ON state. Note that the acquisition function 141 acquires an electric signal indicating that the switch SW1 is in the OFF state and an electric signal indicating that the switch SW2 is in the OFF state.

The determination function 142 determines whether the acquisition function 141 has acquired both the electric signal indicating that the switch SW1 is in the ON state and the electric signal indicating that the switch SW2 is in the ON state.

For example, when the panning knob 5 receives the first operation that is a releasing operation from below, the release function 143 releases the movement restriction of the top plate 32 in the horizontal direction. In the present embodiment, when the panning knob 5 receives the first operation that is a releasing operation from below and the panning knob 5 receives a second operation that is a releasing operation from above, the release function 143 releases the movement restriction of the top plate 32 in the horizontal direction. In other words, when the switch SW2 receives the first input that is an input from below and the switch SW1 receives a second input that is an input from above, the release function 143 releases the movement restriction of the top plate 32 in the horizontal direction. Specifically, when the determination function 142 determines that both the electric signal indicating that the switch SW1 is in the ON state and the electric signal indicating that the switch SW2 is in the ON state have been acquired, the release function 143 releases the movement restriction of the top plate 32 in the horizontal direction by the restriction mechanism 7. In the present embodiment, when the release function 143 releases the movement restriction of the top plate 32 in the horizontal direction, the top plate 32 can be manually moved in the horizontal direction. On the other hand, in other cases, the release function 143 does not release the movement restriction of the top plate 32 in the horizontal direction. That is, when the determination function 142 determines that at least one of the electric signal indicating that the switch SW1 is in the ON state and the electric signal indicating that the switch SW2 is in the ON state is not acquired, the release function 143 does not release the movement restriction of the top plate 32 in the horizontal direction. Furthermore, as described above, in the present embodiment, the physical switch constituting the switch SW2 is a trigger switch. Therefore, the release function 143 releases the movement restriction of the top plate 32 in the horizontal direction on condition that the reception of the first operation, which is a releasing operation from below, with respect to the panning knob 5 is continued. The release function 143 is an example of a releaser in the present embodiment.

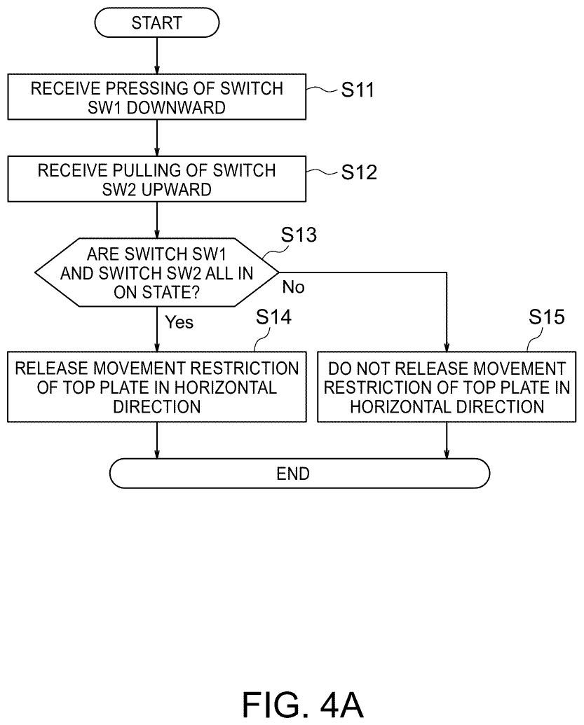

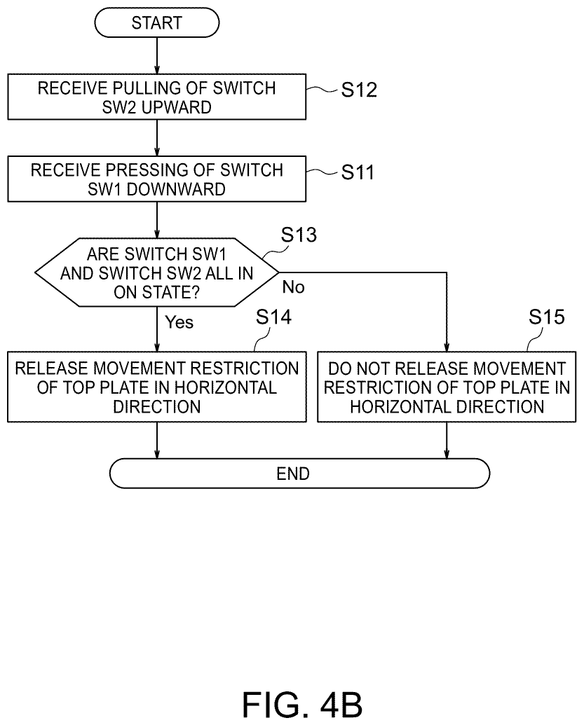

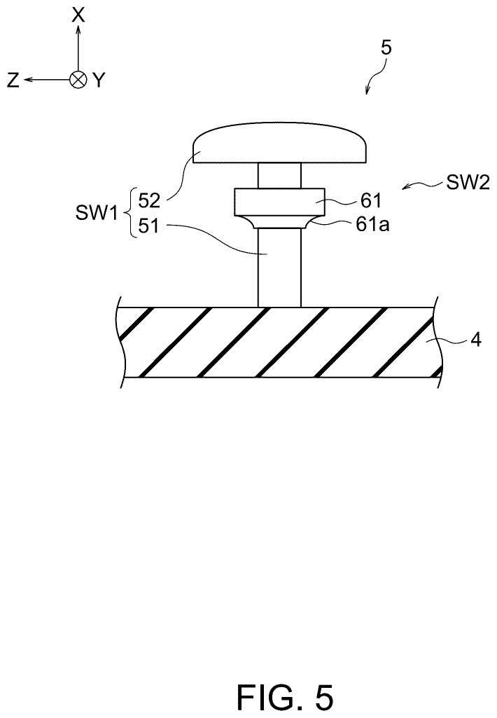

Next, the releasing operation of the X-ray diagnostic apparatus 1 according to the first embodiment configured as described above will be described with reference to FIGS. 4A to 7. FIG. 4A is a flowchart illustrating an example of the releasing operation in the X-ray diagnostic apparatus 1 according to the first embodiment. FIG. 4B is a flowchart illustrating another example of the releasing operation in the X-ray diagnostic apparatus 1 according to the first embodiment. FIGS. 5 to 7 are partial cross-sectional views illustrating the example of the releasing operation in the X-ray diagnostic apparatus 1 according to the first embodiment. This releasing operation is performed when the operator intends to release the movement restriction of the top plate 32 in the horizontal direction. At the start of the releasing operation, the panning knob 5 is positioned in an initial state illustrated in FIG. 5.

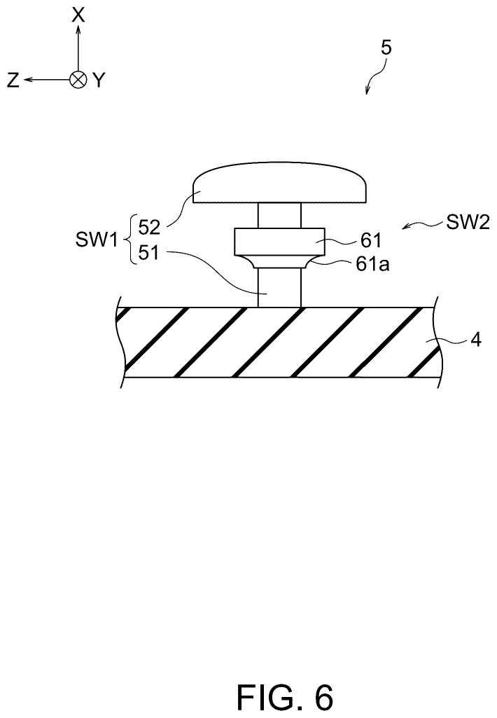

First, as illustrated in FIG. 4A, the acquisition function 141 of the processing circuitry 14 receives pressing of the switch SW1 in the downward direction (step S11). For example, the operator presses the switch SW1 downward. More specifically, for example, the operator presses the palm of the right hand or the left hand against the enlarged portion 52 of the switch SW1 from above, and presses the enlarged portion 52 downward. As a result, as illustrated in FIG. 6, the enlarged portion 52 is moved downward, and the switch SW1 is turned on. The acquisition function 141 of the processing circuitry 14 acquires an electric signal indicating that the switch SW1 is in the ON state. In the example of FIG. 6, the enlarged portion 52 moves downward and the sliding portion 61 moves downward.

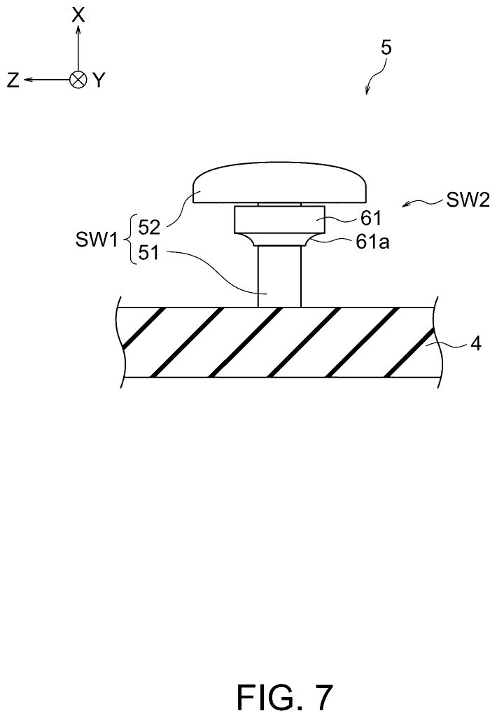

Next, as illustrated in FIG. 4A, the acquisition function 141 of the processing circuitry 14 receives upward pulling of the switch SW2 (step S12). For example, the operator pulls up the switch SW2. For example, the operator grips the sliding portion 61 which is the switch SW2 together with the enlarged portion 52 with a hand pressing the enlarged portion 52, and pulls up the sliding portion 61. As a result, as illustrated in FIG. 7, the sliding portion 61 is moved upward, and the switch SW2 is turned on. In the example of FIG. 7, when the sliding portion 61 is moved in the upward direction, the sliding portion 61 approaches the enlarged portion 52. The acquisition function 141 of the processing circuitry 14 acquires an electric signal indicating that the switch SW2 is in the ON state.

Next, as illustrated in FIG. 4A, the determination function 142 of the processing circuitry 14 determines whether or not both the switch SW1 and the switch SW2 are in the ON state (step S13). That is, the determination function 142 of the processing circuitry 14 determines whether the acquisition function 141 has acquired both the electric signal indicating that the switch SW1 is in the ON state and the electric signal indicating that the switch SW2 is in the ON state.

When the determination function 142 of the processing circuitry 14 determines that both the switch SW1 and the switch SW2 are in the ON state (step S13: Yes), the release function 143 of the processing circuitry 14 releases the movement restriction of the top plate 32 in the horizontal direction by the restriction mechanism 7 (step S14). This enables movement of the top plate 32 in the horizontal direction.

On the other hand, when the determination function 142 of the processing circuitry 14 does not determine that both the switch SW1 and the switch SW2 are in the ON state (step S13: No), that is, when the determination function 142 of the processing circuitry 14 determines that at least one of the electric signal indicating that the switch SW1 is in the ON state and the electric signal indicating that the switch SW2 is in the ON state is not acquired, the release function 143 of the processing circuitry 14 does not release the movement restriction of the top plate 32 in the horizontal direction by the restriction mechanism 7 (step S15). In this case, the top plate 32 cannot move in the horizontal direction.

After step S14 or step S15, the releasing operation of the X-ray diagnostic apparatus 1 ends. In the example of FIG. 4A described above, the downward pressing of the switch SW1 is received in step S11, and then the upward pulling of the switch SW2 is received in step S12. Not limited to this, for example, as illustrated in FIG. 4B, after the upward pulling of the switch SW2 is received in step S12, the downward pressing of the switch SW1 may be received in step S11. In this case, for example, the operator grips the sliding portion 61 which is the switch SW2 together with the enlarged portion 52, pulls up the sliding portion 61, and then presses the enlarged portion 52 of the switch SW1.

According to the X-ray diagnostic apparatus 1 of the first embodiment described above, the release function 143 of the processing circuitry 14 releases the movement restriction of the top plate 32 in the horizontal direction when the switch SW2 detects the first input that is the input from below. Conversely, in a case where the switch SW2 does not detect an input from below, the movement of the top plate 32 in the horizontal direction is restricted. Therefore, even when, for example, the belly of the worker, the arms and legs of the subject P, or a falling object comes into contact with the panning knob 5 from above, the movement of the top plate 32 in the horizontal direction is restricted. As a result, it is possible to reduce an erroneous operation related to the movement of the top plate in the couch 3 of the X-ray diagnostic apparatus 1.

The X-ray diagnostic apparatus 1 according to the present embodiment further includes the switch SW1 that is provided on the panning knob 5 and detects the second input that is the input from above, and the release function 143 of the processing circuitry 14 releases the movement restriction of the top plate 32 in the horizontal direction when both the first input and the second input are detected. As a result, the operational feeling of the switch SW1, which is the conventional operation, can be maintained. In addition, the release of the movement restriction of the top plate 32 in the horizontal direction can be switched by turning on/off the switch SW2 while applying the weight to the switch SW1 and keeping the switch SW1 in the ON state. Therefore, it is not necessary to re-apply the weight as in the case of switching the release of the movement restriction by turning on/off the switch SW1, and fine adjustment of the top plate 32 can be easily performed.

In the present embodiment, the switch SW1 of the panning knob 5 includes the support column 51 extending upward from the operation panel 4, and the enlarged portion 52 provided on the support column 51 and enlarged laterally from the support column 51. As a result, it is possible to maintain the conventional appearance, to be easily recognized by the operator, and to improve operability. For example, by providing the enlarged portion 52, the operator can easily put his/her weight on the panning knob 5. Therefore, even when the subject P is placed on the top plate 32, the top plate 32 can be easily moved. Note that the shape of the panning knob 5 may be a grip shape instead of the shape in which the weight is easily placed or in addition to the shape in which the weight is easily placed.

Further, in the present embodiment, the switch SW1 of the panning knob 5 detects the input by moving the enlarged portion 52 downward. As a result, the operational feeling of the switch SW1, which is the conventional operation, can be maintained.

In addition, in the present embodiment, the switch SW1 and the switch SW2 are configured by trigger switches. As a result, operability of the switch SW1 and the switch SW2 can be improved.

In addition, in the present embodiment, a lower edge portion of the sliding portion 61 of the switch SW2 is provided with the inclined surface 61a directed in a direction opposite to the support column 51 and obliquely downward. As a result, the operator can easily operate the switch SW2. For example, even when the operator grips the panning knob 5 from the lateral direction, the switch SW2 can be easily moved upward.

In the present embodiment, the switch SW1 and the switch SW2 electronically detect an input by the acquisition function 141, the determination function 142, and the release function 143. The present invention is not limited thereto, and the switch SW1 and the switch SW2 may mechanically detect the input. In this case, for example, the restriction mechanism 7 includes a clutch, and when the switch SW1 is moved downward and the switch SW2 is moved upward, the clutch is released. In a case where the switch SW1 and the switch SW2 mechanically detect an input, the acquisition function 141, the determination function 142, and the release function 143 of the processing circuitry 14 may be configured by hardware.

### Second Embodiment

In the first embodiment described above, the switch SW2 is a physical switch that includes the sliding portion 61 and detects an input when the sliding portion 61 is moved upward. However, the configuration of the switch SW2 is not limited to the physical switch. The X-ray diagnostic apparatus according to the second to fourth embodiments in which the configuration of the switch SW2 is changed will be described below.

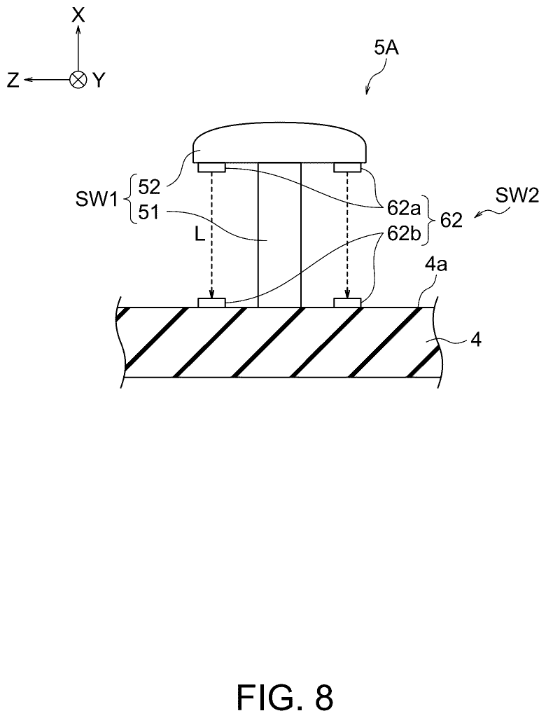

FIG. 8 is a partial cross-sectional view illustrating an example of the operation panel 4 and a panning knob 5A according to the second embodiment. In the present embodiment, the switch SW2 configured by the physical switch in the first embodiment is configured by an optical sensor 62.

As illustrated in FIG. 8, the panning knob 5A includes a light emitter 62a that emits an optical signal L and a light receiver 62b that receives the optical signal L, as the optical sensor 62 that is the switch SW2. The optical signal L is, for example, visible light or laser light. In the present embodiment, in the switch SW2 of the panning knob 5A, the light emitter 62a is provided below the enlarged portion 52 of the switch SW1, and the light receiver 62b is provided facing the light emitter 62a on the operation panel 4. In the example of FIG. 8, the light emitter 62a is provided on the lower surface, that is, the back surface of the enlarged portion 52, and the light receiver 62b is provided on the upper surface 4a of the operation panel 4.

The switch SW2 detects the first input that is an input from below when the light receiver 62b detects a change in the optical signal L. That is, in the present embodiment, the input from below detected by the switch SW2 is an input from below to the light emitter 62a of the switch SW2. In other words, when the light receiver 62b detects a change in the optical signal L, the panning knob 5A receives the first operation from below with respect to the panning knob 5A. For example, the operator holds a finger under the light emitter 62a, that is, between the light emitter 62a and the light receiver 62b. As a result, the optical signal L received by the light receiver 62b is blocked, and the switch SW2 is turned on. When the operator retracts the finger from below the light emitter 62a, the optical signal L received by the light receiver 62b returns to the original state. As a result, the switch SW2 is turned off.

As illustrated in FIG. 8, the switch SW2 may include one or a plurality of light emitters 62a and a plurality of light receivers 62b. In this case, the switch SW2 may switch between the ON state and the OFF state depending on whether or not at least one of the one or a plurality of light receivers 62b detects a change in the optical signal L. Alternatively, the switch SW2 may switch between the ON state and the OFF state depending on whether or not all of the one or the plurality of light receivers 62b detects a change in the optical signal L.

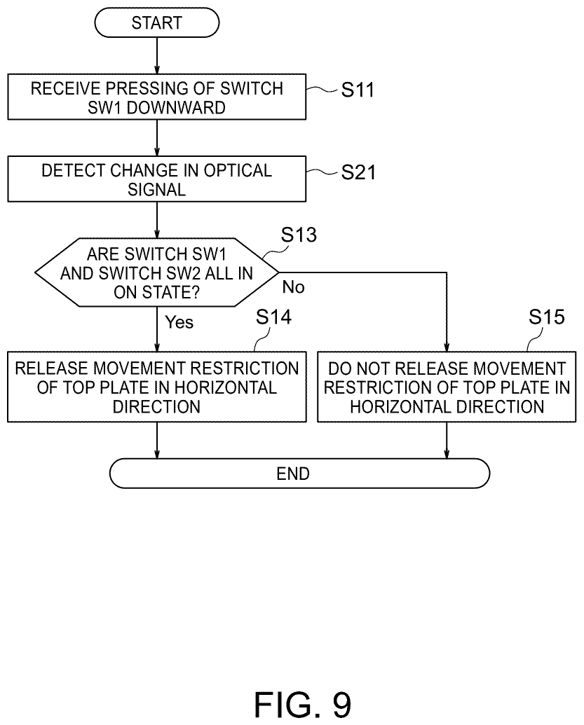

FIG. 9 is a flowchart illustrating an example of a releasing operation in the X-ray diagnostic apparatus according to the second embodiment. As illustrated in FIG. 9, after receiving the pressing of the switch SW1 in the downward direction in step S11, the acquisition function 141 of the processing circuitry 14 detects a change in the optical signal (step S21). For example, after the operator presses the palm of the hand against the enlarged portion 52 from above and presses the enlarged portion 52 downward in step S11, the operator holds the finger of the hand pressing the enlarged portion 52 under the switch SW2. The operator holds his/her finger under the light emitter 62a of the switch SW2 by, for example, making a gripping motion of the enlarged portion 52. As a result, the light receiver 62b detects a change in the optical signal L, and the switch SW2 is turned on. The acquisition function 141 of the processing circuitry 14 acquires an electric signal indicating that the switch SW2 is in the ON state. The subsequent steps are similar to steps S13 to S15 described above.

According to the X-ray diagnostic apparatus 1 of the second embodiment described above, the operation of gripping the enlarged portion 52 is common to the first embodiment, and the difference in operational feeling from the first embodiment can be reduced. In addition, in a case where a drape, a leg, a hand, or the like of the subject P, which reacts to the optical sensor 62, enters under the switch SW2, there is a possibility of causing malfunction, but an unnecessary gripping operation can be omitted as compared with the first embodiment. That is, in the present embodiment, if the weight is put on the panning knob 5A, the releasing operation can be performed without gripping the enlarged portion 52. Therefore, operability of the panning knob 5A can be improved.

Note that the configuration of the optical sensor 62 is not limited to that described above. The first to third modifications of the second embodiment in which the configuration of the optical sensor 62 is changed will be described below. In any of the first to third modifications of the second embodiment, the switch SW2 detects the first input that is an input from below when the light receiver 62b detects a change in the optical signal L.

### First Modification of Second Embodiment

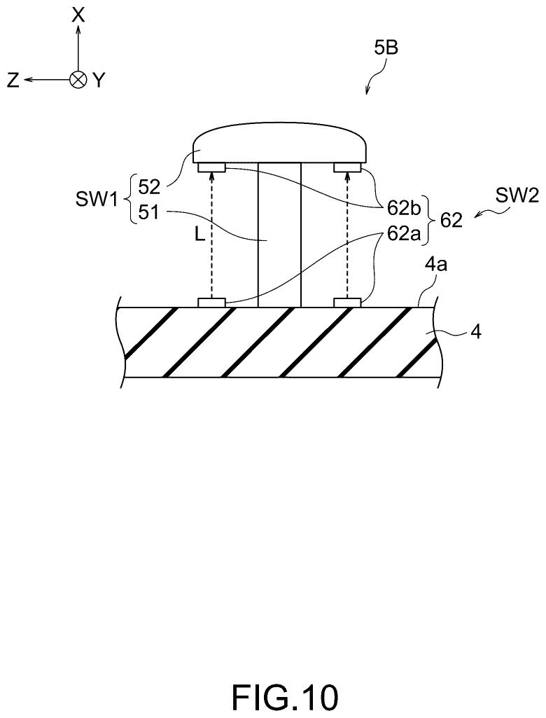

FIG. 10 is a partial cross-sectional view illustrating an example of the operation panel 4 and a panning knob 5B according to Modification 1 of the second embodiment. In the present modification, the positional relationship between the light emitter 62a and the light receiver 62b is opposite to that of the second embodiment.

As illustrated in FIG. 10, in the present modification, in the switch SW2 of the panning knob 5B, the light receiver 62b is provided below the enlarged portion 52 of the switch SW1, and the light emitter 62a is provided facing the light receiver 62b on the operation panel 4. In the example of FIG. 10, the light receiver 62b is provided on the lower surface of the enlarged portion 52, and the light emitter 62a is provided on the upper surface 4a of the operation panel 4. In the present modification, the input from below detected by the switch SW2 is an input from below to the light receiver 62b of the switch SW2. In other words, when the light receiver 62b detects a change in the optical signal L, the panning knob 5B receives the first operation from below with respect to the panning knob 5B.

### Second Modification of Second Embodiment

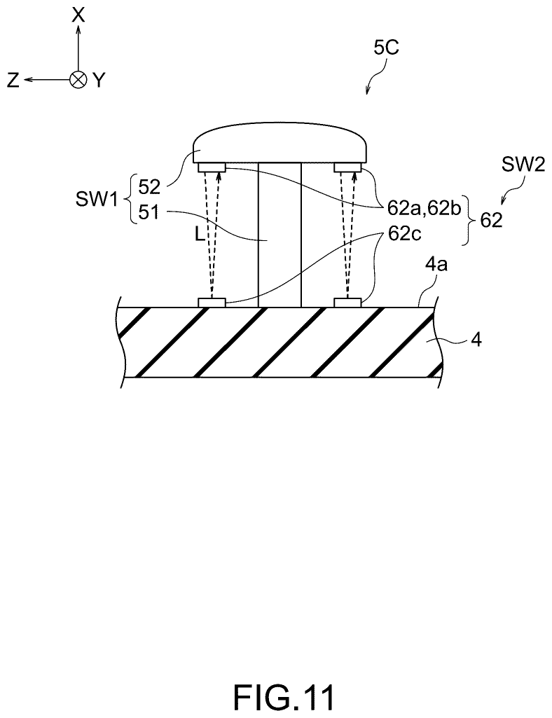

FIG. 11 is a partial cross-sectional view illustrating an example of the operation panel 4 and a panning knob 5C according to the second modification of the second embodiment. In the second embodiment and the first modification of the second embodiment described above, the transmissive optical sensor 62 is provided. On the other hand, in the present modification, a reflective optical sensor 62, that is, a photo-reflector is provided.

In the present modification, the panning knob 5C further includes, as the switch SW2, a reflector 62c that reflects the optical signal L emitted from the light emitter 62a to the light receiver 62b. The reflector 62c is a mirror or the like. The light emitter 62a and the light receiver 62b may be provided in the same unit. As illustrated in FIG. 11, in the switch SW2 of the panning knob 5C, the light emitter 62a and the light receiver 62b are provided below the enlarged portion 52 of the switch SW1, and the reflector 62c is provided facing the light emitter 62a and the light receiver 62b on the operation panel 4. In the example of FIG. 11, the light emitter 62a and the light receiver 62b are provided on the lower surface of the enlarged portion 52, and the reflector 62c is provided on the upper surface 4a of the operation panel 4. Note that instead of providing the reflector 62c, the upper surface 4a of the operation panel 4 may have the function of the reflector 62c. In the present modification, the input from below detected by the switch SW2 is an input from below to the light emitter 62a and the light receiver 62b of the switch SW2. In other words, when the light receiver 62b detects a change in the optical signal L, the panning knob 5C receives the first operation from below with respect to the panning knob 5C.

### Third Modification of Second Embodiment

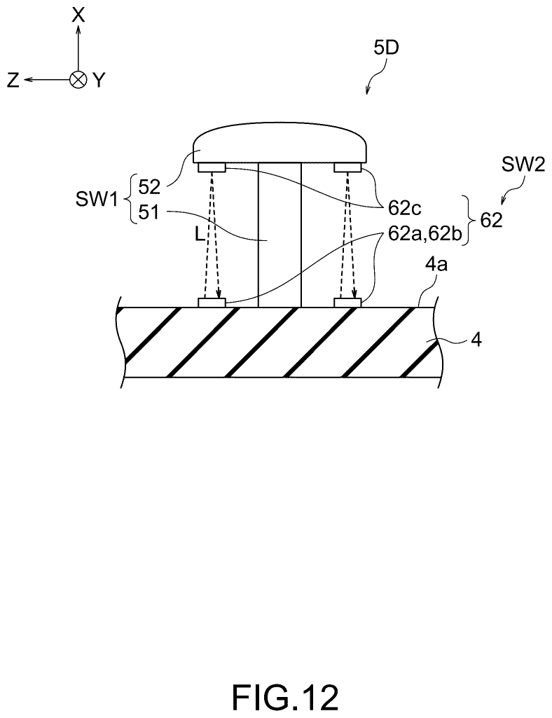

FIG. 12 is a partial cross-sectional view illustrating an example of the operation panel 4 and a panning knob 5D according to the third modification of the second embodiment. In the present modification, the positional relationship between the light emitter 62a and the light receiver 62b, and the reflector 62c is opposite to that of the second modification of the second embodiment.

As illustrated in FIG. 12, in the present modification, in the switch SW2 of the panning knob 5D, the reflector 62c is provided below the enlarged portion 52 of the switch SW1, and the light emitter 62a and the light receiver 62b are provided facing the reflector 62c on the operation panel 4. In the example of FIG. 12, the reflector 62c is provided on the lower surface of the enlarged portion 52, and the light emitter 62a and the light receiver 62b are provided on the upper surface 4a of the operation panel 4. Note that instead of providing the reflector 62c, the lower surface of the enlarged portion 52 may have the function of the reflector 62c. In the present modification, the input from below detected by the switch SW2 is an input from below to the reflector 62c of the switch SW2. In other words, when the light receiver 62b detects a change in the optical signal L, the panning knob 5D receives the first operation from below with respect to the panning knob 5D.

According to the first to third modifications of the second embodiment described above, the degree of freedom in designing the panning knob can be improved.

### Third Embodiment

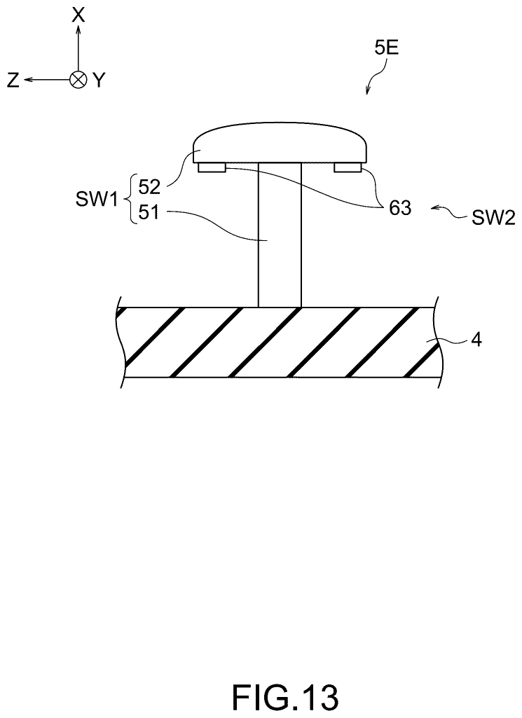

FIG. 13 is a partial cross-sectional view illustrating an example of the operation panel 4 and a panning knob 5E according to the third embodiment. In the present embodiment, the switch SW2 includes a capacitance sensor 63 that is a capacitive touch sensor.

As illustrated in FIG. 13, the panning knob 5E includes, as the switch SW2, the capacitance sensor 63 provided below the enlarged portion 52 of the switch SW1, more specifically, on the lower surface of the enlarged portion 52. The capacitance sensor 63 detects a change in capacitance due to the contact of the operator as the first input that is an input from below. That is, in the present embodiment, the input from below detected by the switch SW2 is an input from below to the capacitance sensor 63 of the switch SW2. In other words, the panning knob 5E receives a change in capacitance due to the contact of the operator with the capacitance sensor 63 as the first operation from below with respect to the panning knob 5E. When the operator comes into contact with the capacitance sensor 63, the capacitance detected by the capacitance sensor 63 changes, and the switch SW2 is turned on. When the operator stops contacting the capacitance sensor 63, the capacitance detected by the capacitance sensor 63 returns to the original value, and the switch SW2 is turned off.

As illustrated in FIG. 13, the switch SW2 of the panning knob 5E may include one or a plurality of capacitance sensors 63. In this case, the switch SW2 may be switched between the ON state and the OFF state depending on whether or not there is a change in capacitance in at least one of the one or the plurality of capacitance sensors 63. Alternatively, the switch SW2 may be switched between the ON state and the OFF state depending on whether or not there is a change in capacitance in all of the one or the plurality of capacitance sensors 63.

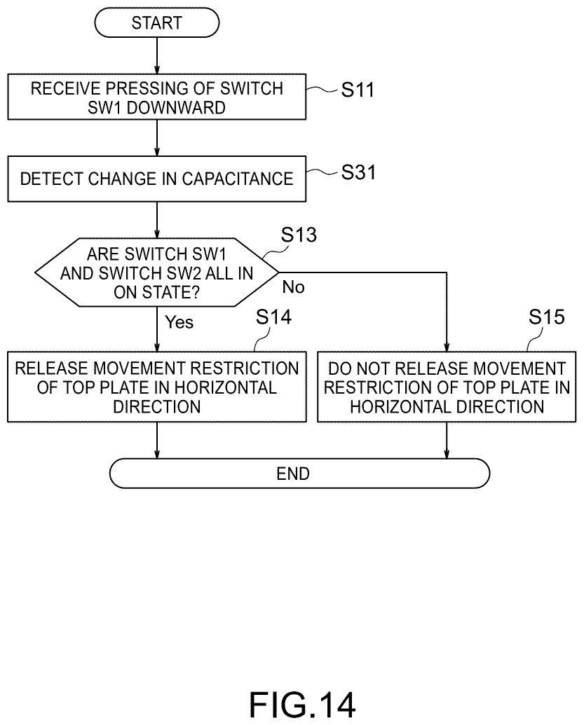

FIG. 14 is a flowchart illustrating an example of a releasing operation in the X-ray diagnostic apparatus according to the third embodiment. As illustrated in FIG. 14, after receiving the pressing of the switch SW1 in the downward direction in step S11, the acquisition function 141 of the processing circuitry 14 detects a change in the capacitance (step S31). For example, in step S11, after the operator presses the palm against the enlarged portion 52 from above and presses the enlarged portion 52 downward, the operator touches the switch SW2 with the fingertip of the hand pressing the enlarged portion 52. As a result, the switch SW2 detects a change in capacitance as the first input that is an input from below, and the switch SW2 is turned on. The acquisition function 141 of the processing circuitry 14 acquires an electric signal indicating that the switch SW2 is in the ON state. The subsequent steps are similar to steps S13 to S15 described above.

According to the X-ray diagnostic apparatus of the third embodiment described above, the operation of gripping the enlarged portion 52 is common to the first embodiment, and the difference in operational feeling from the panning knob 5 of the first embodiment can be reduced. In addition, in a case where an object that reacts to the capacitance sensor 63, such as a drape, a leg, or a hand of the subject P, touches the switch SW2, there is a possibility that malfunction occurs. However, if the user touches the capacitance sensor 63, the panning knob 5E can be turned on without completely gripping the panning knob 5E. Therefore, operability of the panning knob 5E can be improved.

### Fourth Embodiment

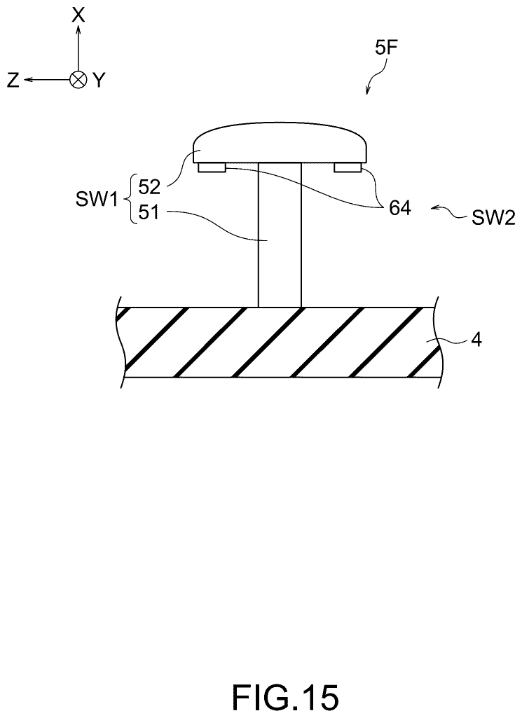

FIG. 15 is a perspective view illustrating an example of the operation panel 4 and a panning knob 5F according to the fourth embodiment. In the present embodiment, the switch SW2 is configured by a pressure sensor 64.

As illustrated in FIG. 15, the panning knob 5F includes, as the switch SW2, the pressure sensor 64 provided below the enlarged portion 52 of the switch SW1, more specifically, on the lower surface of the enlarged portion 52. The pressure sensor 64 detects a change in pressure due to the contact of the operator as the first input that is an input from below. That is, in the present embodiment, the input from below detected by the switch SW2 is an input from below to the pressure sensor 64 of the switch SW2. In other words, the panning knob 5F receives a change in pressure due to the contact of the operator with the pressure sensor 64 as the first operation from below with respect to the panning knob 5F. When the operator comes into contact with the pressure sensor 64, the pressure detected by the pressure sensor 64 changes, and the switch SW2 is turned on. When the operator stops contacting the pressure sensor 64, the pressure detected by the pressure sensor 64 returns to the original value, and the switch SW2 is turned off.

As illustrated in FIG. 15, the switch SW2 may include one or a plurality of pressure sensors 64. In this case, the switch SW2 may be switched between the ON state and the OFF state depending on whether or not there is a change in pressure in at least one of the one or a plurality of pressure sensors 64. Alternatively, the switch SW2 may be switched between the ON state and the OFF state depending on whether or not there is a change in pressure in all of the one or a plurality of pressure sensors 64.

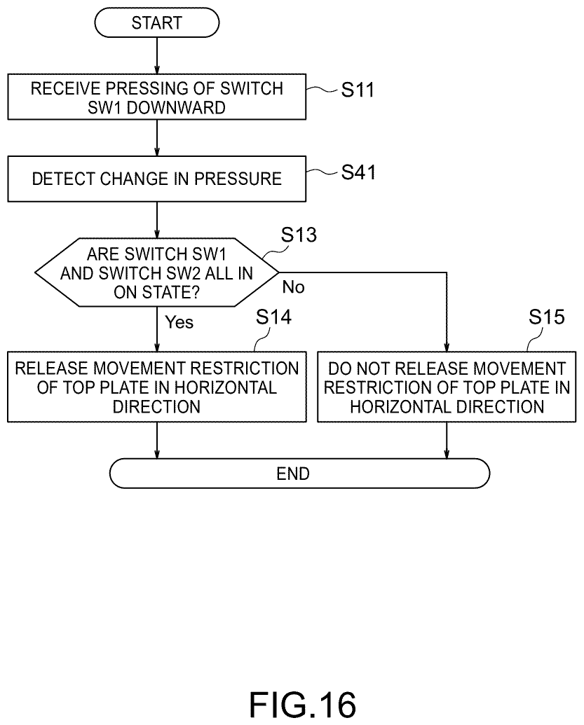

FIG. 16 is a flowchart illustrating an example of a releasing operation in the X-ray diagnostic apparatus according to the fourth embodiment. As illustrated in FIG. 16, after receiving the pressing of the switch SW1 in the downward direction in step S11, the acquisition function 141 of the processing circuitry 14 detects a change in the pressure (step S41). For example, in step S11, after the operator presses the palm against the enlarged portion 52 from above and presses the enlarged portion 52 downward, the operator touches the switch SW2 with the fingertip of the hand pressing the enlarged portion 52. As a result, the switch SW2 detects a change in pressure as the first input that is an input from below, and the switch SW2 is turned on. The acquisition function 141 of the processing circuitry 14 acquires an electric signal indicating that the switch SW2 is in the ON state. The subsequent steps are similar to steps S13 to S15 described above.

According to the X-ray diagnostic apparatus of the fourth embodiment described above, the operation of gripping the enlarged portion 52 is common to the first embodiment, and the difference in operational feeling from the panning knob 5 of the first embodiment can be reduced. In addition, since the pressure sensor 64 itself is small and thin, it can be easily mounted. Further, although the switch SW2 which is a physical switch in the first embodiment has a different mechanism for detecting an input, it is necessary to apply pressure to the pressure sensor 64 to some extent, and thus it is possible to reduce erroneous operations as in the first embodiment.

### Fifth Embodiment

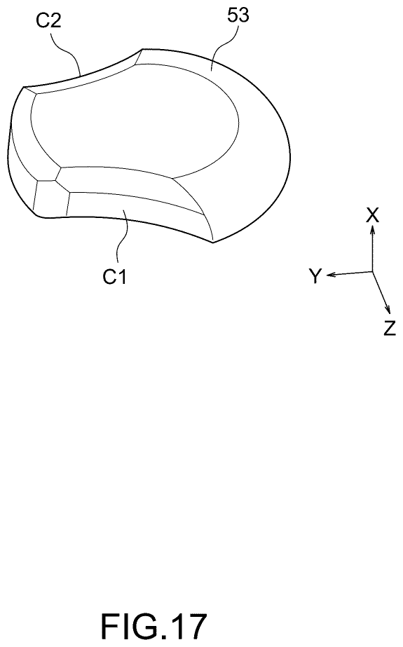

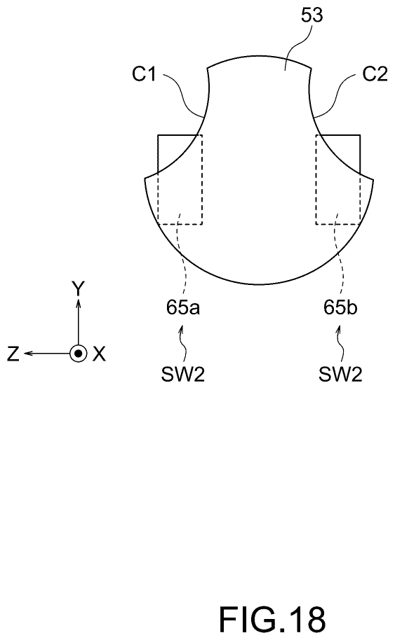

Next, a fifth embodiment in which the shapes of the switch SW1 and the switch SW2 of the panning knob 5 in the first embodiment are changed to shapes that are easier to grip will be described. FIG. 17 is a perspective view illustrating an example of an enlarged portion 53 of the switch SW1 of a panning knob according to the fifth embodiment. FIG. 18 is a plan view illustrating an example of an enlarged portion 53 of the switch SW1 and the switch SW2 of a panning knob according to the fifth embodiment.

As illustrated in FIGS. 17 and 18, the enlarged portion 53 of the switch SW1 of the panning knob according to the present embodiment has a planar shape including two recesses C1 and C2. The number of recesses is not limited to two and may be any number. That is, the enlarged portion 53 of the switch SW1 may have a planar shape including one or a plurality of recesses.

As illustrated in FIG. 18, a physical switch 65a and a physical switch 65b that receive an operation from below with respect to the panning knob are provided as the switch SW2 of the panning knob according to the present embodiment below each of the recesses in the enlarged portion 53. In the example of FIG. 18, the physical switch 65a is provided below recess C1 on the lower surface of enlarged portion 53, and the physical switch 65b is provided below the recess C2 on the lower surface of the enlarged portion 53. Both the physical switch 65a and the physical switch 65b are examples of a first receiver in the present embodiment. In the present embodiment, both the physical switch 65a and the physical switch 65b are trigger-type switches that detect an input by being moved upward. The number of switches SW2 is not limited to two and may be any number. That is, one or a plurality of switches SW2 may be provided below one or a plurality of recesses in the enlarged portion 53.

The two switches SW2 illustrated in FIG. 18 detect an input from below to at least one of the switches SW2 as the first input. In this manner, the one or plurality of switches SW2 detect an input from below to at least one of the one or plurality of switches SW2 as the first input. In other words, the panning knob according to the present embodiment receives the first operation from below on the panning knob 5 by moving at least one of the one or plurality of switches SW2 in the upward direction. When at least one of the one or plurality of switches SW2 detects the first input, the one or plurality of switches SW2 are turned on, and an electric signal indicating the ON state is output to the control device 10.

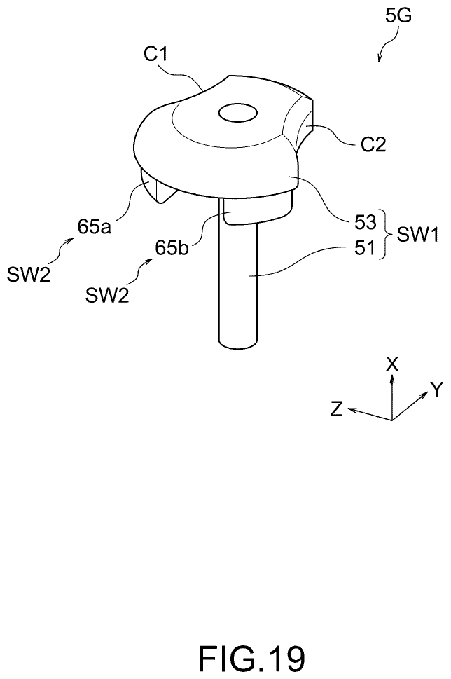

Note that, in the example of FIG. 18, the physical switch 65a and the physical switch 65b have rectangular planar shapes. The present invention is not limited thereto, and as illustrated in FIG. 19, the physical switch 65a and the physical switch 65b may have shapes along the shapes of the side portions of the enlarged portion 53.

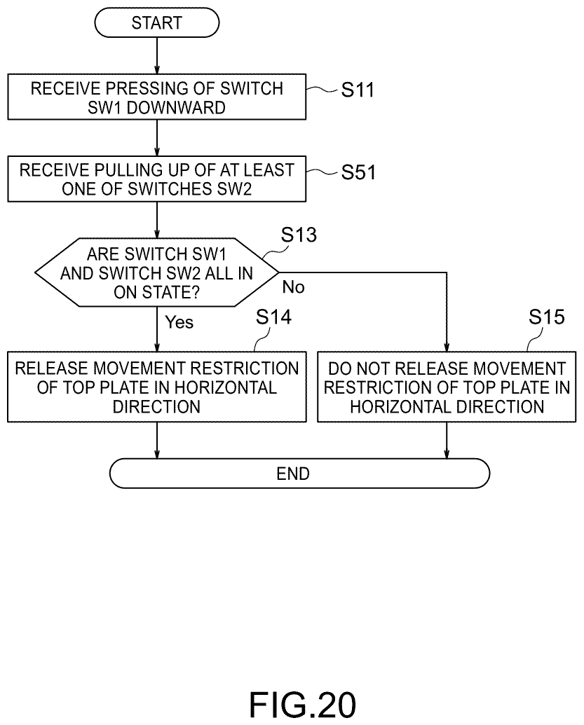

FIG. 20 is a flowchart illustrating an example of a releasing operation in the X-ray diagnostic apparatus according to the fifth embodiment. As illustrated in FIG. 20, after receiving the pressing of the switch SW1 in the downward direction in step S11, the acquisition function 141 of the processing circuitry 14 receives the pulling up of at least one of the switches SW2 (step S51). For example, in step S11, after the operator presses the palm against the enlarged portion 52 from above and presses the enlarged portion 52 downward, the operator pulls up at least one of the switches SW2 by the fingertip of the hand pressing the enlarged portion 52. In the examples of FIGS. 18 and 19, the operator pulls up at least one of the physical switch 65a and the physical switch 65b.

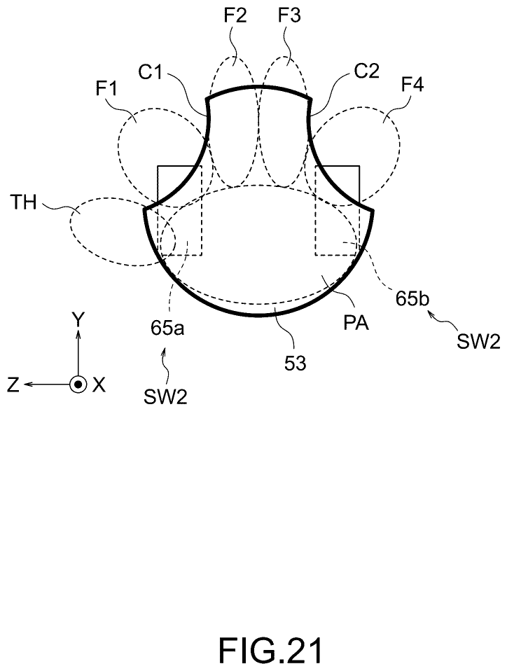

In step S51, the operator's hand is arranged as illustrated in FIG. 21, for example. FIG. 21 is a plan view illustrating an example of the releasing operation in the X-ray diagnostic apparatus according to the fifth embodiment. FIG. 21 illustrates a palm portion PA, a thumb TH, an index finger F1, a middle finger F2, a ring finger F3, and a little finger F4 of the operator. The operator presses the palm portion PA against the enlarged portion 53 from above, and grips the side portion of the enlarged portion 53 with the thumb TH, the middle finger F2, and the ring finger F3. Then, the operator pulls up at least one of the physical switch 65a and the physical switch 65b with at least one of the index finger F1 and the little finger F4. As a result, two switches SW2 are turned on. The acquisition function 141 of the processing circuitry 14 acquires an electric signal indicating that the switch SW2 is in the ON state. The subsequent steps are similar to steps S13 to S15 described above.

According to the X-ray diagnostic apparatus according to the fifth embodiment described above, since the enlarged portion 53 of the switch SW1 and the switch SW2 have a shape that is easier to grip, the operability of the panning knob can be improved.

### Sixth Embodiment

The panning knob according to each of the above-described embodiments and modifications includes two switches, that is, the switch SW1 and the switch SW2. On the other hand, the switch SW1 may be a dummy from the viewpoint that it is sufficient that the switch SW1 has a structure in which a weight is applied to the switch SW1 can be gripped. Hereinafter, a sixth embodiment in which the switch SW1 in the first embodiment is a dummy will be described.

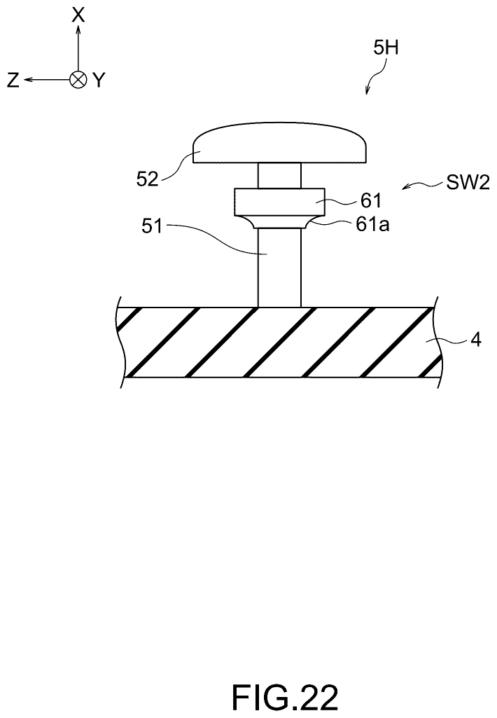

FIG. 22 is a perspective view illustrating an example of the operation panel 4 and a panning knob 5H according to the sixth embodiment. The panning knob 5H of the present embodiment does not include the switch SW1 and does not detect an input from above. The panning knob 5H is immovably fixed to the operation panel 4 when the panning knob 5H is operated from above. More specifically, the panning knob 5H includes the support column 51 extending upward from the operation panel 4, and the enlarged portion 52 provided on the support column 51 and enlarged laterally from the support column 51. Here, the support column 51 is immovably fixed to the operation panel 4, and the enlarged portion 52 is immovably fixed to the support column 51.

On the other hand, the switch SW2 of the panning knob 5H according to the present embodiment detects an input from below as in the first embodiment. More specifically, the panning knob 5H includes, as the switch SW2, a sliding portion 61 provided so as to be slidable on the support column 51 of the panning knob 5H, and detects the first input that is the input from below by the sliding portion 61 being moved in the upward direction. In other words, the panning knob 5H receives the first operation from below with respect to the panning knob 5H when the sliding portion 61 is moved upward.

In the present embodiment, when the panning knob 5H receives the first operation that is a releasing operation from below, the release function 143 of the processing circuitry 14 releases the movement restriction of the top plate 32 in the horizontal direction. In other words, when the switch SW2 receives the first input from below, the release function 143 releases the movement restriction of the top plate 32 in the horizontal direction. Specifically, when the determination function 142 of the processing circuitry 14 determines that the first input is detected by the switch SW2, the release function 143 releases the movement restriction of the top plate 32 in the horizontal direction. Furthermore, similarly to the first embodiment, in the present embodiment, the physical switch constituting the switch SW2 is a trigger switch. Therefore, the release function 143 releases the movement restriction of the top plate 32 in the horizontal direction on condition that the reception of the first operation, which is a releasing operation from below, with respect to the panning knob 5H is continued.

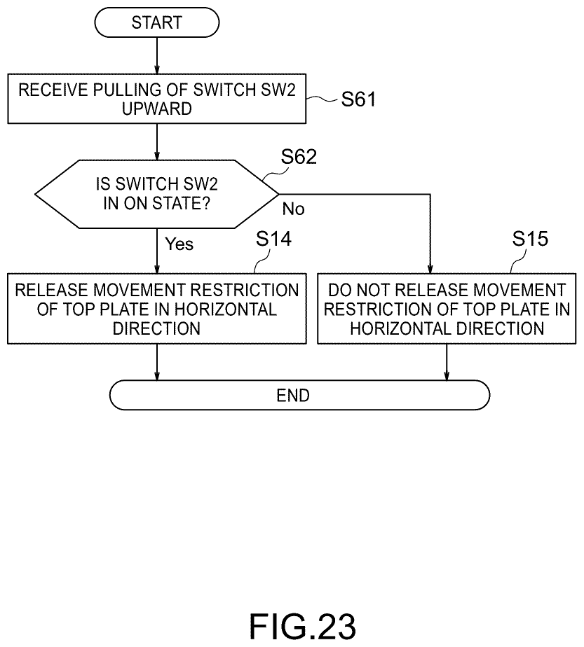

FIG. 23 is a flowchart illustrating an example of a releasing operation in the X-ray diagnostic apparatus according to the sixth embodiment. At the start of the releasing operation, the panning knob 5H is positioned in an initial state illustrated in FIG. 22.

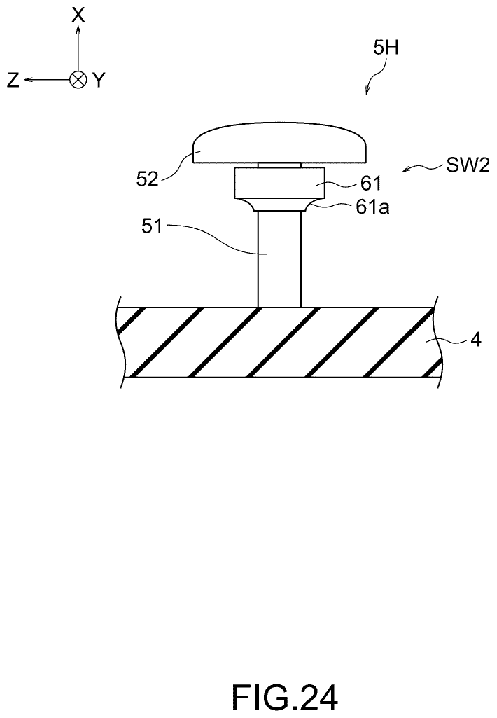

First, as illustrated in FIG. 23, the acquisition function 141 of the processing circuitry 14 receives upward pulling of the switch SW2 (step S61). For example, the operator pulls up the switch SW2. More specifically, for example, the operator puts the palm of the right hand or the left hand on the enlarged portion 52 from above and grips the enlarged portion 52. Then, the operator pulls up the sliding portion 61 with the fingertip of the hand gripping the enlarged portion 52. As a result, as illustrated in FIG. 24, the sliding portion 61 constituting the switch SW2 is moved upward, and the switch SW2 is turned on. The acquisition function 141 of the processing circuitry 14 acquires an electric signal indicating that the switch SW2 is in the ON state.

Next, as illustrated in FIG. 23, the determination function 142 of the processing circuitry 14 determines whether the switch SW2 is in the ON state (step S62). That is, the determination function 142 of the processing circuitry 14 determines whether the electric signal indicating that the switch SW1 is in the ON state has been acquired by the acquisition function 141 of the processing circuitry 14.

When it is determined that the switch SW2 is in the ON state (step S62: Yes), the release function 143 of the processing circuitry 14 releases the movement restriction of the top plate 32 in the horizontal direction by the restriction mechanism 7 (step S14). This enables movement of the top plate 32 in the horizontal direction.

On the other hand, when it is not determined that the switch SW2 is in the ON state (step S62: No), the release function 143 of the processing circuitry 14 does not release the movement restriction of the top plate 32 in the horizontal direction by the restriction mechanism 7 (step S15). In this case, the top plate 32 cannot move in the horizontal direction.

After step S14 and step S15, the releasing operation of the X-ray diagnostic apparatus ends.

In the X-ray diagnostic apparatus 1 according to the sixth embodiment described above, the movement restriction of the top plate 32 in the horizontal direction is released by pulling up one switch SW2. However, since the operation of pulling up the switch SW2 is less likely to occur by chance, even if the input necessary for the releasing operation is one input of the switch SW2, the risk of an erroneous operation due to pressing of the operator's belly or the like can be reduced. In addition, by making the switch SW1 dummy, it is possible to reduce an erroneous operation in which the switch SW1 is pressed due to pressing of the operator's belly or dropping of an object. Furthermore, by making it a dummy, the operator can operate the panning knob 5H with a feeling close to the conventional operational feeling.

In the present embodiment, the sliding portion 61, which is a physical switch similar to that of the first embodiment, is used as the switch SW2. The switch SW2 is not limited thereto, and an optical sensor 62, a capacitance sensor 63, or a pressure sensor 64 similar to those of the second to fourth embodiments may be used. When the pressure sensor 64 that is the switch SW2 of the fourth embodiment is used, the risk of erroneous operation of the switch SW2 can be further reduced.

In addition, instead of the enlarged portion 52 of the present embodiment, the enlarged portion 53 of the fifth embodiment may be provided, and one or a plurality of switches SW2 may be provided. As a result, the enlarged portion 53 and the switch SW2 have a shape that is easier to grip, so that the operability of the panning knob can be improved.

In the present embodiment, the panning knob 5H is immovably fixed to the operation panel 4. The present invention is not limited thereto, and the panning knob 5H may be configured not to detect an input from above although it is not immovably fixed to the operation panel 4.

### Seventh Embodiment

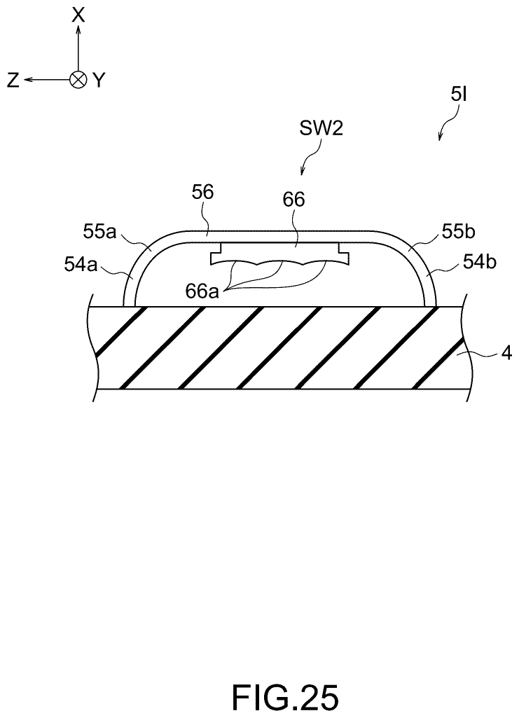

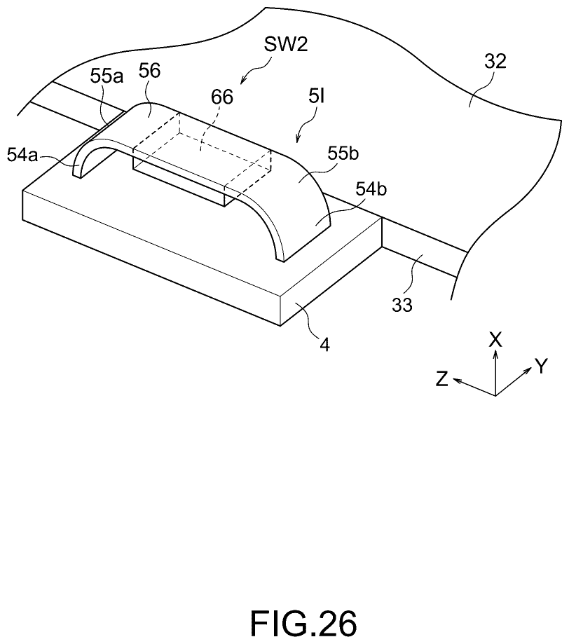

The shape of the dummy is not limited to the support column 51 and the enlarged portion 52 in the sixth embodiment described above. Hereinafter, a seventh embodiment will be described in which an inverted U-shaped dummy is provided and the switch SW2 is provided inside so that the panning knob can be easily gripped. FIG. 25 is a perspective view illustrating an example of the operation panel 4 and a panning knob 5I according to the seventh embodiment. FIG. 26 is a perspective view illustrating an example of the top plate 32 and the operation panel 4 according to the seventh embodiment.

As illustrated in FIGS. 25 and 26, the panning knob 5I of the present embodiment includes an upright portion 54a provided upright from the operation panel 4, an upright portion 54b provided upright from the operation panel 4 and separated from the upright portion 54a, and a suspending portion 56 suspended between an upper portion of the upright portion 54a and an upper portion of the upright portion 54b. The upright portion 54a is an example of a first upright portion in the present embodiment. The upright portion 54b is an example of a second upright portion in the present embodiment. Note that, in the examples of FIGS. 25 and 26, the suspending portion 56 is suspended from the upright portion 54a via the connection portion 55a, and the suspending portion 56 is suspended from the upright portion 54a via the connection portion 55b. Here, both the connection portion 55a and the connection portion 55b have a rounded shape having a curved surface.

The panning knob 5I is immovably fixed to the operation panel 4 when the panning knob 5I is operated from above. More specifically, the upright portion 54a and the upright portion 54b are immovably fixed to the operation panel 4, and the suspending portion 56 is immovably fixed to the upright portion 54a and the upright portion 54b.

In the present embodiment, the switch SW2 of the panning knob 5I is a trigger type physical switch 66. As illustrated in FIGS. 25 and 26, the switch SW2 of the panning knob 5I is provided below the suspending portion 56, and is moved in the upward direction to detect the first input that is the input from below. That is, in the present embodiment, the input from below detected by the switch SW2 is an input from below to the physical switch 66 which is the switch SW2. In other words, the panning knob 5I receives the first operation from below with respect to the panning knob 5I when the physical switch 66 is moved upward. The physical switch 66 is an example of a second receiver in the present embodiment. Note that the switch SW2 of the second to fourth embodiments may be provided as the switch SW2.

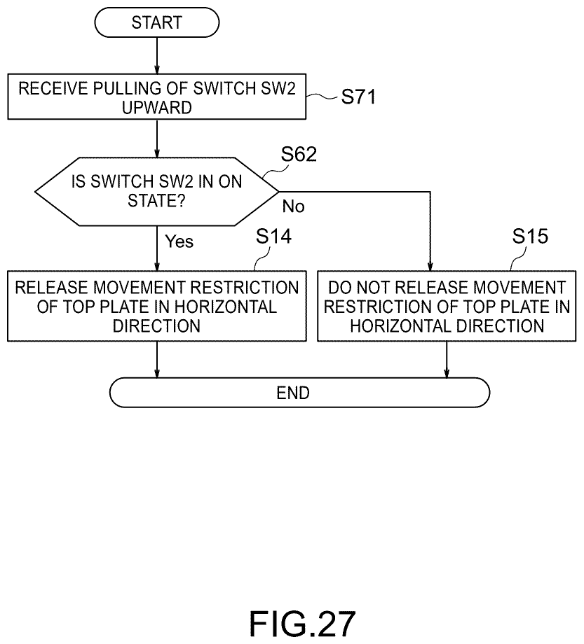

FIG. 27 is a flowchart illustrating an example of a releasing operation in the X-ray diagnostic apparatus according to the seventh embodiment.

First, as illustrated in FIG. 27, the acquisition function 141 of the processing circuitry 14 receives upward pulling of the switch SW2 (step S71). For example, the operator pulls up the switch SW2. More specifically, for example, the operator puts the palm of the right hand or the left hand on the suspending portion 56 from above and holds the suspending portion 56 and the physical switch 66 with the hand. As a result, the switch SW2 is turned on. The acquisition function 141 of the processing circuitry 14 acquires an electric signal indicating that the switch SW2 is in the ON state. The subsequent steps are similar to steps S62 and S14 to S15 described above.

According to the X-ray diagnostic apparatus of the seventh embodiment described above, since the suspending portion 56 is supported by the upright portion 54a and the upright portion 54b, the stability of the suspending portion 56 can be improved, and the operability of the panning knob 5I can be improved.

In the present embodiment, the suspending portion 56 is suspended from the upright portion 54a via the connection portion 55a, the suspending portion 56 is suspended from the upright portion 54a via the connection portion 55b, and both the connection portion 55a and the connection portion 55b have curved surfaces. By providing the connection portion 55a and the connection portion 55b, the weight can be easily applied to the panning knob 5I, and the operability can be improved.

As illustrated in FIG. 26, the direction from the upright portion 54a to the upright portion 54b of the panning knob 5I may be along the longitudinal direction of the couch, that is, the Z-axis direction. This makes it possible to reduce the size of protrusion of the panning knob 5I from the couch. Also, the operator can intuitively hold the panning knob 5I. That is, the operator can grip the panning knob 5I without twisting the hand, and the operability can be improved.

As illustrated in FIG. 25, the recess 66a may be provided below the physical switch 66. In the example of FIG. 25, three recesses 66a are provided. Thus, the physical switch 66 can be easily gripped. The number of recesses 66a is not limited to three and may be any number.

### Eighth Embodiment

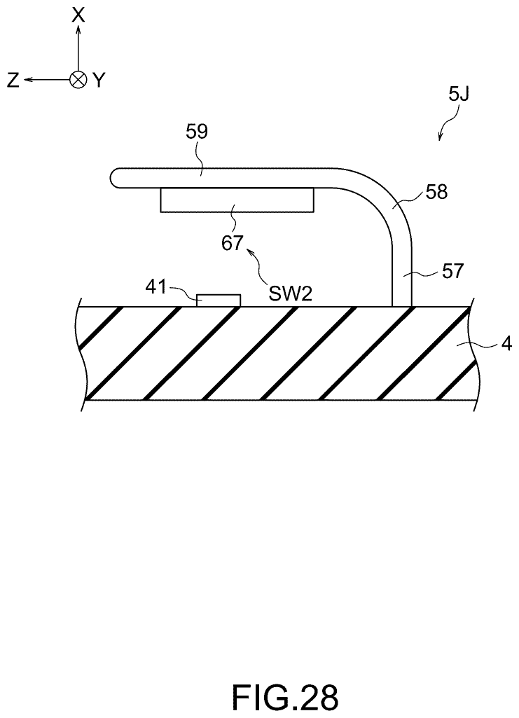

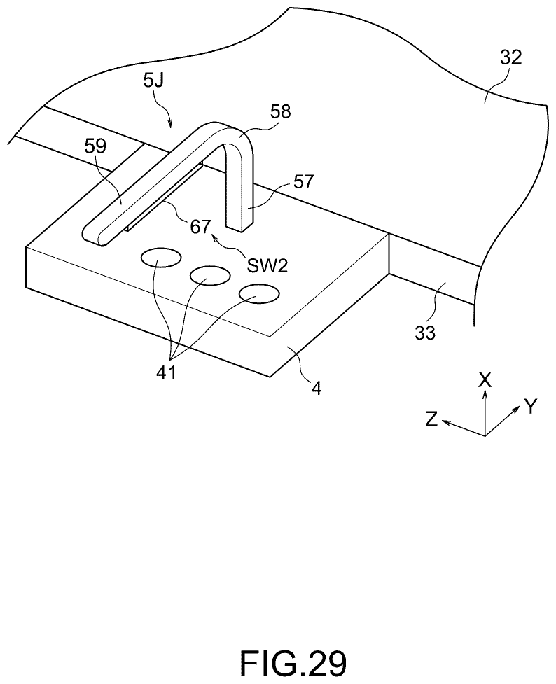

Next, an eighth embodiment will be described in which an L-shaped dummy is provided and the switch SW2 is provided inside so that the panning knob can be easily gripped. FIG. 28 is a perspective view illustrating an example of the operation panel 4 and a panning knob 5J according to the eighth embodiment. FIG. 29 is a perspective view illustrating an example of the top plate 32 and the operation panel 4 according to the eighth embodiment.

As illustrated in FIGS. 28 and 29, the panning knob 5J of the present embodiment includes an upright portion 57 provided upright from the operation panel 4, and a lateral extension portion 59 having one end connected to an upper portion of the upright portion 57, extending laterally from the one end, and having the other end serving as a free end. The upright portion 57 is an example of a third upright portion in the present embodiment. In the examples of FIGS. 27 and 28, one end of the lateral extension portion 59 is connected to the upper portion of the upright portion 57 via a connection portion 58 having a rounded shape. An operation button 41 of the operation panel 4 is provided below the lateral extension portion 59.

The panning knob 5J is immovably fixed to the operation panel 4 when the panning knob 5J is operated from above. More specifically, the upright portion 57 is immovably fixed to the operation panel 4, and the lateral extension portion 59 is immovably fixed to the upright portion 57.

In the present embodiment, the switch SW2 of the panning knob 5J is a trigger type physical switch 67. As illustrated in FIGS. 27 and 28, the switch SW2 of the panning knob 5J is provided below the lateral extension portion 59, and is moved upward to detect the first input that is the input from below. That is, in the present embodiment, the input from below detected by the switch SW2 is an input from below to the physical switch 67 which is the switch SW2. In other words, the panning knob 5J receives the first operation from below with respect to the panning knob 5J when the physical switch 67 is moved upward. The physical switch 67 is an example of a third receiver in the present embodiment.

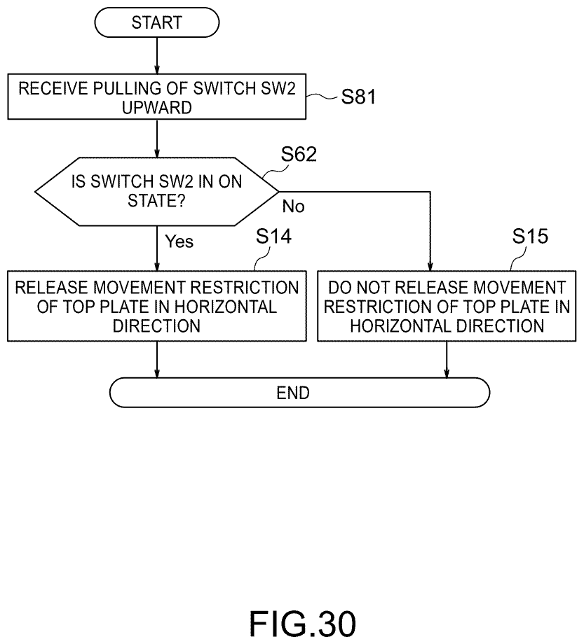

FIG. 30 is a flowchart illustrating an example of a releasing operation in the X-ray diagnostic apparatus according to the eighth embodiment.

First, as illustrated in FIG. 30, the acquisition function 141 of the processing circuitry 14 receives upward pulling of the switch SW2 (step S81). For example, the operator pulls up the switch SW2. More specifically, for example, the operator puts the palm of the right hand or the left hand on the lateral extension portion 59 from above and holds the lateral extension portion 59 and the physical switch 67 with the hand. As a result, the switch SW2 is turned on. The acquisition function 141 of the processing circuitry 14 acquires an electric signal indicating that the switch SW2 is in the ON state. The subsequent steps are similar to steps S62 and S14 to S15 described above.

According to the X-ray diagnostic apparatus of the eighth embodiment described above, since the other end of the lateral extension portion 59 is the free end, the lateral extension portion 59 can be easily gripped.

As illustrated in FIG. 29, the direction in which the lateral extension portion 59 extends may be orthogonal to the longitudinal direction of the top plate 32. That is, the direction from one end of the lateral extension portion 59 connected to the upper portion of the upright portion 57 to the other end of the lateral extension portion 59 may be along the Y-axis direction. As a result, the weight can be easily applied to the lateral extension portion 59, and the operability of the panning knob 5J can be improved.

In addition, in the present embodiment, the operation button 41 is provided below the lateral extension portion 59. In this manner, the operation panel 4 can be made compact by disposing the operation button 41 and the like in an empty space below the lateral extension portion 59.

The upright portion 57, the connection portion 58, and the lateral extension portion 59 may be integrally molded. Accordingly, the stability of the panning knob 5J can be improved. In addition, the panning knob 5J can be easily installed on the operation panel 4.

### Ninth Embodiment

In the first embodiment described above, the movement of the top plate 32 in the horizontal direction is performed by human power. The present invention is not limited thereto, and the top plate 32 may be moved in the horizontal direction by electric power.

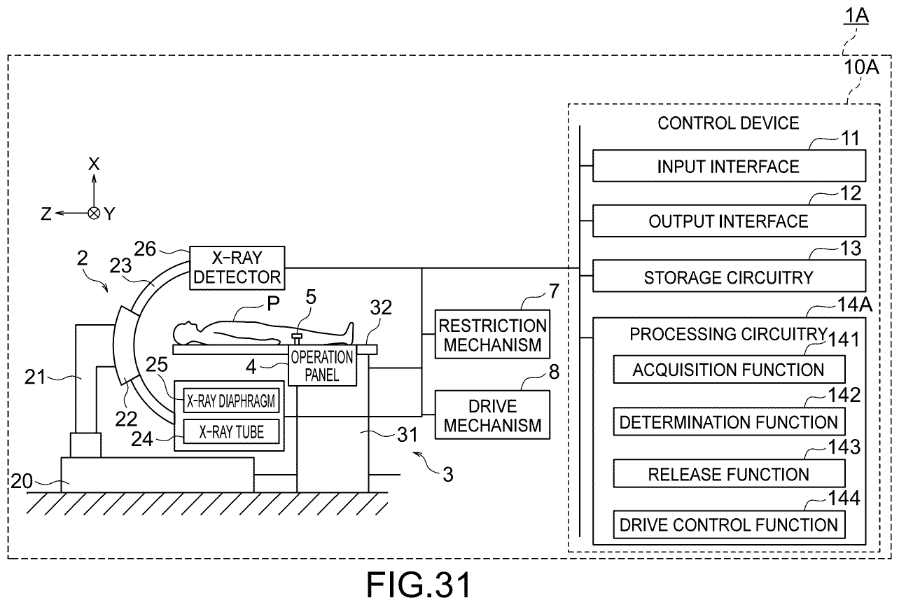

FIG. 31 is a block diagram illustrating a configuration example of an X-ray diagnostic apparatus 1A according to a ninth embodiment. As illustrated in FIG. 31, in the present embodiment, a processing circuitry 14A of a control device 10A further includes a drive control function 144 in addition to the configuration of the processing circuitry 14 according to the first embodiment.

The drive control function 144 controls the drive mechanism 8 based on an operator's instruction. The drive control function 144 is an example of a drive control unit in the present embodiment. In the present embodiment, when the release function 143 of the processing circuitry 14A releases the movement restriction of the top plate 32 in the horizontal direction, the top plate 32 can be controlled to move in the horizontal direction by the drive control function 144.

The drive mechanism 8 of the present embodiment drives the movement of the top plate 32 in the horizontal direction. For example, the drive mechanism 8 includes motors mounted on the longitudinal and lateral shafts of the top plate 32.

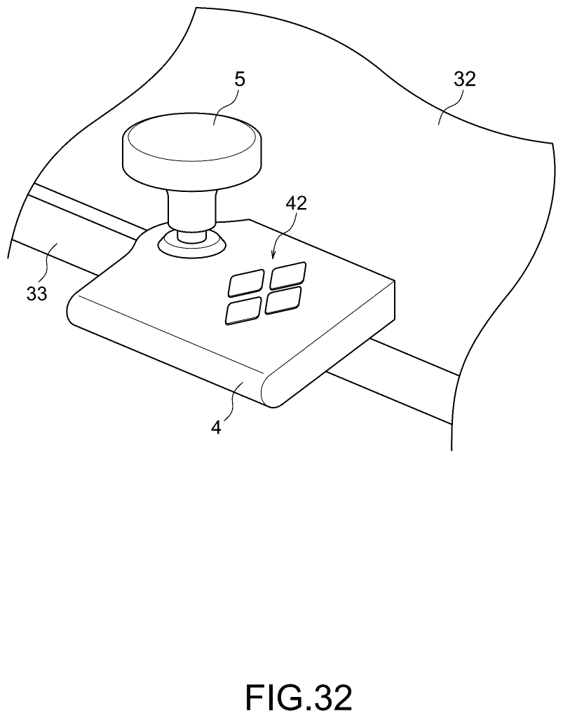

FIG. 32 is a perspective view illustrating an example of the top plate 32 and the operation panel 4 according to the ninth embodiment. The operation panel 4 of the present embodiment is provided with an operation button 42 for the operator to instruct the top plate 32 to move in the horizontal direction. The operator operates the operation button 42 while releasing the movement restriction of the top plate 32 in the horizontal direction by operating the panning knob 5. The drive control function 144 controls the drive mechanism 8 based on an operator's instruction input to the operation button 42. When the drive mechanism 8 is driven, the top plate 32 is moved in the horizontal direction. Instead of the panning knob 5, the panning knobs 5A to 5J of the second to eighth embodiments described above may be provided on the operation panel 4.

According to the X-ray diagnostic apparatus 1A according to the ninth embodiment described above, since the top plate 32 is electrically moved in the horizontal direction, it is possible to reduce the burden on the operator.

The operation button 42 for operating the movement of the top plate 32 in the horizontal direction may be provided on an operation panel different from the operation panel 4 provided with the panning knob 5. Alternatively, the operation button 42 may be provided on a remote controller or the like. This can improve the operability of the horizontal movement of the top plate 32.

### Tenth Embodiment

Instead of moving the top plate 32 in the horizontal direction by human power, a power assist function for assisting the movement of the top plate 32 in the horizontal direction may be provided.

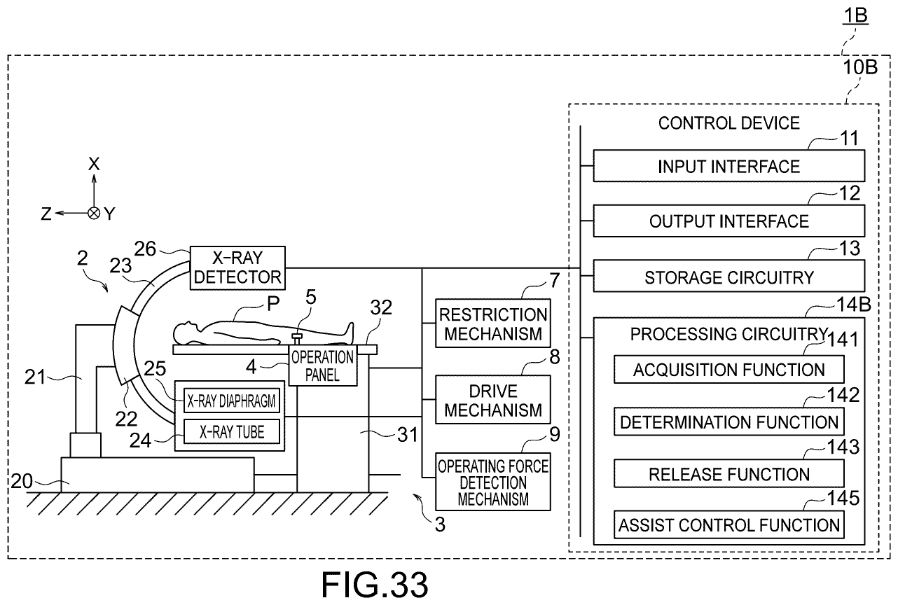

FIG. 33 is a block diagram illustrating a configuration example of an X-ray diagnostic apparatus 1B according to a tenth embodiment. As illustrated in FIG. 33, the X-ray diagnostic apparatus 1B of the present embodiment further includes an operating force detection mechanism 9 in addition to the configuration of the X-ray diagnostic apparatus 1 of the first embodiment. Further, in the present embodiment, a processing circuitry 14B of a control device 10B further includes an assist control function 145 in addition to the configuration of the processing circuitry 14 of the first embodiment. Further, the drive mechanism 8 of the present embodiment drives the movement of the top plate 32 in the horizontal direction.

The operating force detection mechanism 9 detects the operating force in the horizontal direction with respect to the panning knob 5. The operating force detection mechanism 9 is an example of an operating force sensor in the present embodiment. The operating force detection mechanism 9 includes, for example, a contact sensor provided on the support column 51 of the panning knob 5. The operating force detection mechanism 9 may detect the operating force in the horizontal direction with respect to the top plate 32 instead of the operating force in the horizontal direction with respect to the panning knob 5 or in addition to the operating force in the horizontal direction with respect to the panning knob 5. Instead of the panning knob 5, the panning knobs 5A to 5J of the second to eighth embodiments described above may be provided.

The assist control function 145 supports the movement of the top plate 32 in the horizontal direction according to the horizontal operating force detected by the operating force detection mechanism 9. Specifically, the assist control function 145 controls the drive mechanism 8 to apply, to the top plate 32, a driving force in the same direction as the operating force in the horizontal direction detected by the operating force detection mechanism 9 and a constant multiple of the operating force, for example. The assist control function 145 is an example of an assist control unit in the present embodiment.

In the present embodiment, when the release function 143 releases the movement restriction of the top plate 32 in the horizontal direction, the top plate 32 can be manually moved in the horizontal direction and the assist control function 145 supports the movement of the top plate 32 in the horizontal direction.

According to the X-ray diagnostic apparatus 1B according to the tenth embodiment described above, since the top plate 32 is assisted in moving in the horizontal direction, it is possible to reduce the burden on the operator.

The X-ray diagnostic apparatus may be switchable among a case where the movement of the top plate 32 in the horizontal direction is performed by human power, a case where the movement is performed by electric power, and a case where the movement is assisted.

### Eleventh Embodiment

In each of the above-described embodiments, the movement restriction of the top plate 32 in the horizontal direction may be released on condition that the presence of the user is further detected. The user is, for example, an operator of the top plate 32 described above. Hereinafter, an eleventh embodiment including a mechanism for detecting the presence of a user will be described focusing on differences from the sixth embodiment described above.

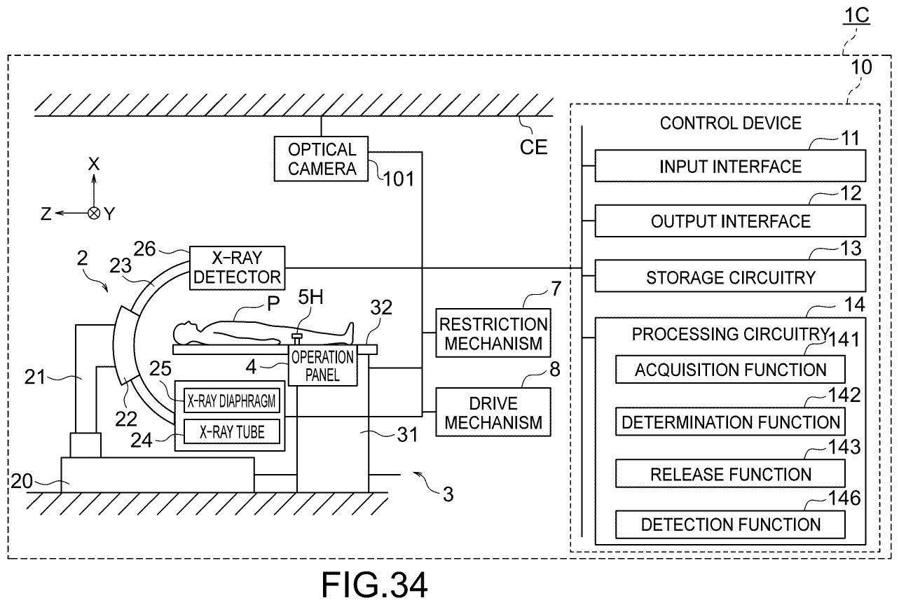

FIG. 34 is a block diagram illustrating a configuration example of an X-ray diagnostic apparatus 1C according to the eleventh embodiment. As illustrated in FIG. 34, the X-ray diagnostic apparatus 1C according to the present embodiment includes the panning knob 5H according to the sixth embodiment. The X-ray diagnostic apparatus 1C according to the present embodiment includes an optical camera 101 in addition to the configuration of the X-ray diagnostic apparatus 1 of the first embodiment. Further, in the present embodiment, the processing circuitry 14 of the control device 10 further includes a detection function 146 in addition to the configuration of the processing circuitry 14 of the first embodiment.

The optical camera 101 is an optical imaging device that detects light, converts the light into an electric signal, and acquires optical information. For example, the optical camera 101 captures an image of a region including the operation panel 4 and acquires optical information on the region. In the example of FIG. 34, the optical camera 101 is an optical camera provided on a ceiling CE of an inspection room. The present invention is not limited thereto, and the optical camera 101 may be an optical camera provided on a side wall or the like of the inspection room, or may be an optical camera that can be mounted on a part of the body of the user, such as an optical camera provided on a wearable device such as a smart glass.

The detection function 146 detects the presence of the user based on the optical information acquired using the optical camera 101. For example, the detection function 146 determines whether or not a marker is included in the optical information related to the region including the operation panel 4, thereby detecting whether the user exists in front of the panning knob 5H. As a method of detecting the presence of the user, for example, there is a method of attaching a marker to the user, and determining whether information regarding the marker is included in the acquired optical information by the detection function 146. As a result, the detection function 146 can distinguish between the user who operates the panning knob 5H and another person such as an assistant around the subject P or the couch 3. As another example, the detection function 146 may detect the presence of the user based on the acquired optical information using a training model such as segmentation. Also in this case, the user and another person may be distinguishable from each other. The detection function 146 is an example of a detection unit in the present embodiment.

In the present embodiment, the release function 143 releases the movement restriction of the top plate 32 in the horizontal direction when the panning knob 5H receives the first operation that is a releasing operation from below and the detection function 146 detects that the user exists in front of the panning knob 5H.

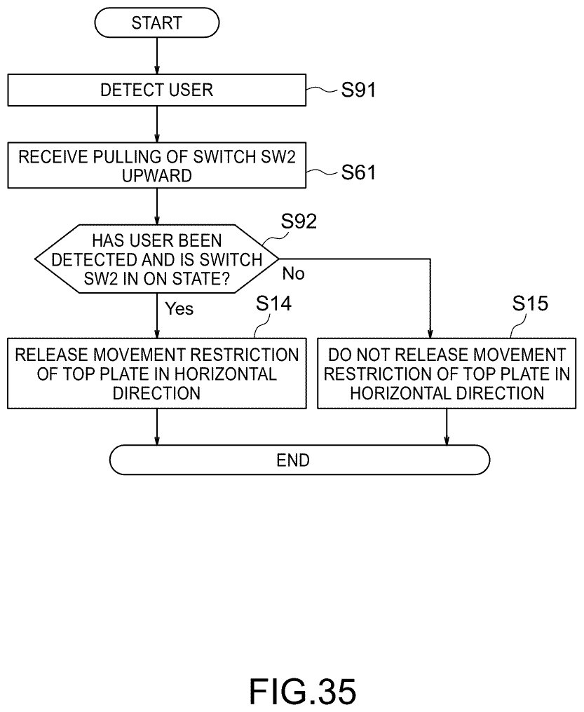

Next, the releasing operation of the X-ray diagnostic apparatus 1C configured as described above will be described with reference to FIG. 35. FIG. 35 is a flowchart illustrating an example of a releasing operation in the X-ray diagnostic apparatus 1C according to the eleventh embodiment.

First, as illustrated in FIG. 35, the detection function 146 of the processing circuitry 14 detects a user (step S91). More specifically, the operator moves in front of the panning knob 5H to operate the panning knob 5H. The detection function 146 detects whether the user exists in front of the panning knob 5H based on the optical information acquired using the optical camera 101. Next step S61 is the same as that in the sixth embodiment described above.

After step S61, the determination function 142 of the processing circuitry 14 determines the user has been detected and whether the switch SW2 is in the ON state (step S92). That is, the determination function 142 of the processing circuitry 14 determines whether or not both the information indicating that the user exists in front of the panning knob 5H by the detection function 146 of the processing circuitry 14 and the electric signal indicating that the switch SW2 is in the ON state have been acquired by the acquisition function 141 of the processing circuitry 14.

When the determination function 142 of the processing circuitry 14 determines that the user has been detected and the switch SW2 are in the ON state (step S92: Yes), the release function 143 of the processing circuitry 14 releases the movement restriction of the top plate 32 in the horizontal direction by the restriction mechanism 7 (step S14).

On the other hand, when the determination function 142 of the processing circuitry 14 does not determine that the user has been detected and the switch SW2 is in the ON state (step S92: No), that is, when the determination function 142 of the processing circuitry 14 determines that at least one of the information indicating that the user exists in front of the panning knob 5H and the electric signal indicating that the switch SW2 is in the ON state is not acquired, the release function 143 of the processing circuitry 14 does not release the movement restriction of the top plate 32 in the horizontal direction by the restriction mechanism 7 (step S15).

After step S14 or step S15, the releasing operation of the X-ray diagnostic apparatus 1C ends.

According to the X-ray diagnostic apparatus 1C of the eleventh embodiment described above, when the panning knob includes one switch SW2, the malfunction can be further reduced. Specifically, it is possible to reduce malfunction when the user is not present in front of the panning knob 5H. Furthermore, in a case where the detection function 146 of the processing circuitry 14 can distinguish between the user and another person, it is possible to further reduce malfunction when a person other than the user approaches the panning knob 5H.

In addition, by automatically detecting the user with the optical camera, a part of the releasing operation is automated as compared with the case of including the two switches SW1 and SW2 as in the first to fifth, ninth, and tenth embodiments, and the operability of the panning knob 5H can be improved.

The present embodiment may be combined with the panning knobs 5I and 5J according to the seventh or eighth embodiment including one switch SW2 instead of the panning knob 5H of the sixth embodiment.

### Modification of Eleventh Embodiment

In addition, the eleventh embodiment described above may be combined with any of the first to fifth, ninth, and tenth embodiments in which the panning knob includes two switches SW1 and SW2. Hereinafter, a modification of the eleventh embodiment in which the eleventh embodiment is combined with the first embodiment will be described focusing on differences from the eleventh embodiment described above.

In the present modification, the panning knob 5 illustrated in FIG. 1 and the like is provided instead of the panning knob 5H illustrated in FIG. 34. In addition, when the panning knob 5 receives the first operation that is a releasing operation from below and a second operation from above with respect to the panning knob 5, and the detection function 146 detects that the user exists in front of the panning knob 5, the release function 143 releases the movement restriction of the top plate 32 in the horizontal direction.

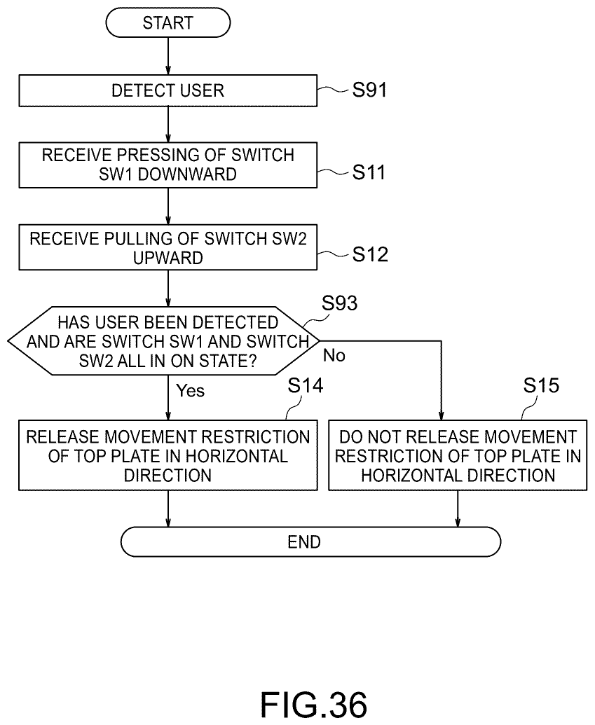

FIG. 36 is a flowchart illustrating an example of a releasing operation in the X-ray diagnostic apparatus according to the modification of the eleventh embodiment. Next step S91 of FIG. 36 is the same as that in the eleventh embodiment described above. Also, steps S11 to S12 are similar to those in the first embodiment described above.

As illustrated in FIG. 36, after step S12, the determination function 142 of the processing circuitry 14 determines the user has been detected and whether or not both the switch SW1 and the switch SW2 are in the ON state (step S93). That is, the determination function 142 of the processing circuitry 14 determines whether or not the information indicating that the user exists in front of the panning knob 5H by the detection function 146 of the processing circuitry 14, the electric signal indicating that the switch SW1 is in the ON state, and the electric signal indicating that the switch SW2 is in the ON state have been acquired by the acquisition function 141 of the processing circuitry 14.

When the determination function 142 of the processing circuitry 14 determines that the user has been detected and both the switch SW1 and the switch SW2 are in the ON state (step S93: Yes), the release function 143 of the processing circuitry 14 releases the movement restriction of the top plate 32 in the horizontal direction by the restriction mechanism 7 (step S14).

On the other hand, when the determination function 142 of the processing circuitry 14 does not determine that the user has been detected and both the switch SW1 and the switch SW2 are in the ON state (step S93: No), that is, when the determination function 142 of the processing circuitry 14 determines that at least one of the information indicating that the user exists in front of the panning knob 5H, the electric signal indicating that the switch SW1 is in the ON state, and the electric signal indicating that the switch SW2 is in the ON state is not acquired, the release function 143 of the processing circuitry 14 does not release the movement restriction of the top plate 32 in the horizontal direction by the restriction mechanism 7 (step S15).

After step S14 and step S15, the releasing operation of the X-ray diagnostic apparatus ends.

According to the X-ray diagnostic apparatus according to the modification of the eleventh embodiment described above, when the panning knob includes the two switches SW1 and SW2, the malfunction can be further reduced. Specifically, it is possible to reduce malfunction when the user is not present in front of the panning knob 5.

### Twelfth Embodiment

In each of the above-described embodiments, the movement restriction of the top plate 32 in the horizontal direction may be released on condition that a voice command is input. Hereinafter, a twelfth embodiment including a mechanism for inputting a voice command will be described focusing on differences from the sixth embodiment described above.

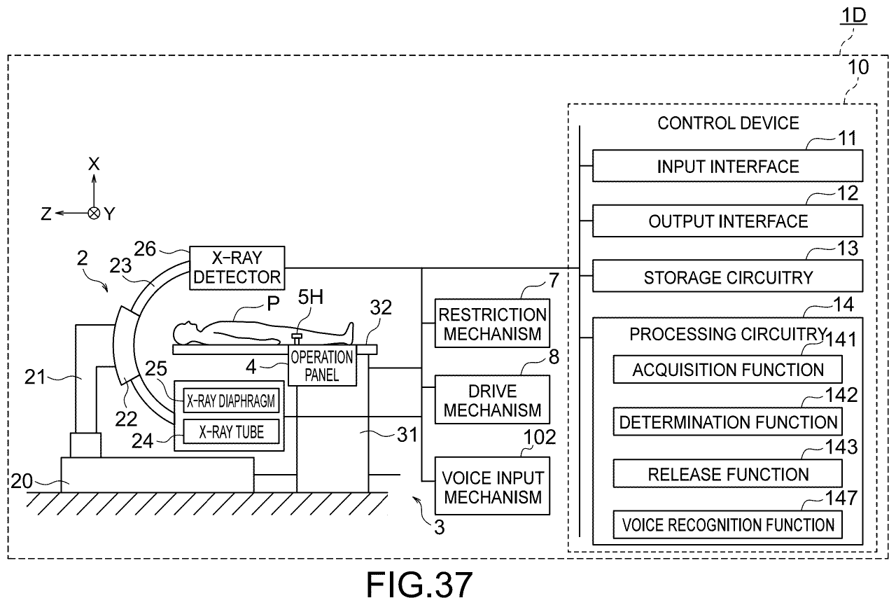

FIG. 37 is a block diagram illustrating a configuration example of an X-ray diagnostic apparatus 1D according to the twelfth embodiment. As illustrated in FIG. 37, the X-ray diagnostic apparatus 1D according to the present embodiment includes the panning knob 5H according to the sixth embodiment. The X-ray diagnostic apparatus 1D according to the present embodiment includes a voice input mechanism 102 in addition to the configuration of the X-ray diagnostic apparatus 1 according to the first embodiment. Further, in the present embodiment, the processing circuitry 14 of the control device 10 further includes a voice recognition function 147 in addition to the configuration of the processing circuitry 14 of the first embodiment.

The voice input mechanism 102 is an interface to which a voice is input from the user. The voice input mechanism 102 may be a part of the input interface 11. The voice input mechanism 102 is implemented by a microphone, a voice input circuitry, and the like. The voice input mechanism 102 may be provided on the operation panel 4 or may be attachable to a part of the body of the user. Alternatively, the voice input mechanism 102 may be implemented by a terminal such as a smartphone or a tablet of the user. The voice input mechanism 102 is an example of a voice inputter in the present embodiment.

The voice recognition function 147 recognizes a voice command from the user based on voice information acquired using the voice input mechanism 102. The voice recognition function 147 determines whether or not a voice command for releasing the movement restriction has been input from the user. The voice command for releasing the movement restriction is, for example, a prescribed voice such as “unlock couch” uttered by the user. Furthermore, for example, voice recognition function 147 distinguishes whether the input voice is a voice related to a voice command or another voice. Note that voice recognition function 147 may be able to distinguish whether the input voice is the voice of the user operating panning knob 5H or the voice of another person other than the user. The voice recognition function 147 is an example of a voice recognition unit in the present embodiment.

In the present embodiment, when the panning knob 5H receives the first operation that is a releasing operation from below and the voice recognition function 147 receives an input of a voice command for releasing the movement restriction, the release function 143 releases the movement restriction of the top plate 32 in the horizontal direction.

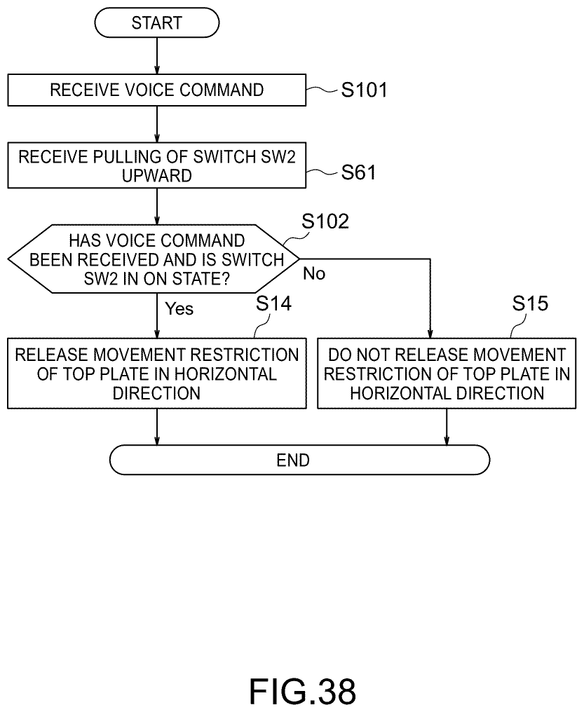

FIG. 38 is a flowchart illustrating an example of a releasing operation in the X-ray diagnostic apparatus 1D according to the twelfth embodiment.

First, as illustrated in FIG. 38, the voice recognition function 147 of the processing circuitry 14 receives a voice command (step S101). More specifically, in order to operate the panning knob 5H, the operator utters a voice related to a voice command for releasing the movement restriction to the voice input mechanism 102. The voice recognition function 147 recognizes whether or not a voice command for releasing the movement restriction has been input from the user based on the voice information input using the voice input mechanism 102. Next step S61 is the same as that in the sixth embodiment described above.

After step S61, the determination function 142 of the processing circuitry 14 determines the voice command has been received and whether the switch SW2 is in the ON state (step S102). That is, the determination function 142 of the processing circuitry 14 determines whether both the information indicating that the voice command has been received by the voice recognition function 147 of the processing circuitry 14 and the electric signal indicating that the switch SW2 is in the ON state have been acquired by the acquisition function 141 of the processing circuitry 14.

When the determination function 142 of the processing circuitry 14 determines that the voice command has been received and the switch SW2 are in the ON state (step S102: Yes), the release function 143 of the processing circuitry 14 releases the movement restriction of the top plate 32 in the horizontal direction by the restriction mechanism 7 (step S14).

On the other hand, when the determination function 142 of the processing circuitry 14 does not determine that the voice command has been received and the switch SW2 is in the ON state (step S102: No), that is, when the determination function 142 of the processing circuitry 14 determines that at least one of the information indicating that the voice command has been received and the electric signal indicating that the switch SW2 is in the ON state is not acquired, the release function 143 of the processing circuitry 14 does not release the movement restriction of the top plate 32 in the horizontal direction by the restriction mechanism 7 (step S15).

After step S14 and step S15, the releasing operation of the X-ray diagnostic apparatus ends.

According to the X-ray diagnostic apparatus 1D of the twelfth embodiment described above, when the panning knob includes one switch SW2, the malfunction can be further reduced. Specifically, it is possible to reduce malfunction when no voice command is input. Furthermore, by the voice recognition function 147 of the processing circuitry 14 distinguishing whether the voice is a voice related to a voice command or another voice, it is possible to reduce malfunctions due to statements other than the voice command. Furthermore, in a case where the voice recognition function 147 of the processing circuitry 14 can distinguish between the user's voice and the voice of another person, it is possible to reduce malfunction due to the voice uttered by a person other than the user.

In addition, by accepting one releasing operation by a voice command, the user's hand can be made free as compared with the case where two switches SW1 and SW2 are provided as in the first to fifth, ninth, and 10 embodiments, and the operability of the panning knob 5H can be improved.

The present embodiment may be combined with the panning knobs 5I and 5J according to the seventh or eighth embodiment including one switch SW2 instead of the panning knob 5H of the sixth embodiment.

### Modification of Twelfth Embodiment

In addition, the twelfth embodiment described above may be combined with any of the first to fifth, ninth, and tenth embodiments in which the panning knob includes two switches SW1 and SW2. Hereinafter, a modification of the twelfth embodiment in which the twelfth embodiment is combined with the first embodiment will be described focusing on differences from the twelfth embodiment described above.

In the present modification, the panning knob 5 illustrated in FIG. 1 and the like is provided instead of the panning knob 5H illustrated in FIG. 37. In addition, when the panning knob 5 receives the first operation that is a releasing operation from below and a second operation from above with respect to the panning knob 5, and the voice recognition function 147 receives an input of a voice command for releasing the movement restriction, the release function 143 releases the movement restriction of the top plate 32 in the horizontal direction.

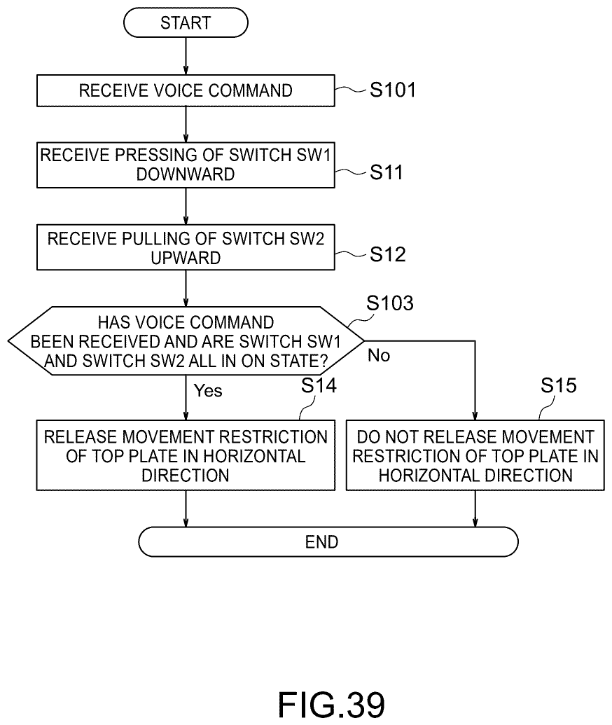

FIG. 39 is a flowchart illustrating an example of a releasing operation in the X-ray diagnostic apparatus according to the modification of the twelfth embodiment. Next step S101 of FIG. 39 is the same as that in the twelfth embodiment described above. Also, steps S11 to S12 are similar to those in the first embodiment described above.

As illustrated in FIG. 39, after step S12, the determination function 142 of the processing circuitry 14 determines the voice command has been received and whether or not both the switch SW1 and the switch SW2 are in the ON state (step S103). That is, the determination function 142 of the processing circuitry 14 determines whether or not the information indicating that the voice command has been received by the voice recognition function 147 of the processing circuitry 14, the electric signal indicating that the switch SW1 is in the ON state, and the electric signal indicating that the switch SW2 is in the ON state have been acquired by the acquisition function 141 of the processing circuitry 14.

When the determination function 142 of the processing circuitry 14 determines that the voice command has been received and both the switch SW1 and the switch SW2 are in the ON state (step S103: Yes), the release function 143 of the processing circuitry 14 releases the movement restriction of the top plate 32 in the horizontal direction by the restriction mechanism 7 (step S14).

On the other hand, when the determination function 142 of the processing circuitry 14 does not determine that the voice command has been received and both the switch SW1 and the switch SW2 are in the ON state (step S103: No), that is, when the determination function 142 of the processing circuitry 14 determines that at least one of the information indicating that voice command for releasing the movement restriction has been received, the electric signal indicating that the switch SW1 is in the ON state, and the electric signal indicating that the switch SW2 is in the ON state is not acquired, the release function 143 of the processing circuitry 14 does not release the movement restriction of the top plate 32 in the horizontal direction by the restriction mechanism 7 (step S15).

After step S14 and step S15, the releasing operation of the X-ray diagnostic apparatus ends.

According to the X-ray diagnostic apparatus 1 according to the modification of the twelfth embodiment described above, when the panning knob includes the two switches SW1 and SW2, the malfunction can be further reduced. Specifically, it is possible to reduce malfunction when no voice command is input.

The term “processor” used herein refers to, for example, a central processing unit (CPU) or a graphics processing unit (GPU), or a circuit such as an application-specific integrated circuit (ASIC), a programmable logic device (such as a simple programmable logic device (SPLD)), a complex programmable logic device (CPLD), a field programmable gate array (FPGA)), etc. In the case of the processor being a CPU, for example, the processor realizes a function by reading and executing a program stored in a storage circuit. On the other hand, in the case of the processor being, for example, an ASIC, instead of a program being stored in a storage circuit, a corresponding function is directly incorporated as a logic circuit in the circuit of the processor. Each processor of the present embodiment is not limited to a configuration as a single circuit; a plurality of independent circuits may be combined into one processor to realize the function of the processor. Furthermore, multiple components or features may be integrated as one processor to realize the respective functions. Furthermore, a plurality of component elements in FIG. 1 may be integrated into one processor to realize the functions.

According to at least one embodiment described above, it is possible to reduce an erroneous operation related to top plate movement in a medical bed device such as an X-ray diagnostic apparatus.

While certain embodiments and their modifications have been described, these embodiments and modifications have been presented by way of example only, and are not intended to limit the scope of the inventions. Indeed, a variety of other forms such as omissions, substitutions, changes and combinations may be made in the embodiments and modifications without departing from the spirit of the inventions. Such embodiments and modifications are intended to be covered by the scope and spirit of the inventions as well as the claimed inventions and their equivalents.

1. An X-ray diagnostic apparatus, comprising:
a restrictor configured to set a movement restriction of a top plate in a horizontal direction, wherein a subject is placed on the top plate;
a manipulator configured to receive a releasing operation for releasing the movement restriction; and
a releaser configured to release the movement restriction when the manipulator receives a first operation that is the releasing operation from below.

2. The X-ray diagnostic apparatus of claim 1, wherein the releaser is configured to release the movement restriction when the manipulator receives the first operation and the manipulator receives a second operation that is the releasing operation from above.

3. The X-ray diagnostic apparatus of claim 1, wherein the releaser is configured to release the movement restriction on condition that a reception of the first operation is continued.

4. The X-ray diagnostic apparatus of claim 2, wherein the manipulator comprises a support column extending upward from an operation panel, and an enlarged portion provided on the support column and enlarged laterally from the support column, and the manipulator receives the second operation when the enlarged portion is moved downward.

5. The X-ray diagnostic apparatus of claim 4, wherein the manipulator further comprises a sliding portion provided to be slidable on the support column, and the manipulator receives the first operation when the sliding portion is moved in an upward direction.

6. The X-ray diagnostic apparatus of claim 4, wherein the manipulator further comprises a light emitter that emits an optical signal and a light receiver that receives the optical signal, and the manipulator receives the first operation when the light receiver detects a change in the optical signal.

7. The X-ray diagnostic apparatus of claim 6, wherein in the manipulator,
the light emitter is provided below the enlarged portion, and the light receiver is provided to face the light emitter on the operation panel; or
the light receiver is provided below the enlarged portion, and the light emitter is provided to face the light receiver on the operation panel.

8. The X-ray diagnostic apparatus of claim 6, wherein the manipulator further comprises a reflector that reflects the optical signal emitted from the light emitter to the light receiver, and
in the manipulator,
the light emitter and the light receiver are provided below the enlarged portion, and the reflector is provided to face the light emitter and the light receiver on the operation panel, or
the reflector is provided below the enlarged portion, and the light emitter and the light receiver are provided to face the reflector on the operation panel.

9. The X-ray diagnostic apparatus of claim 6, wherein the optical signal is visible light or laser light.

10. The X-ray diagnostic apparatus of claim 4, wherein the manipulator further comprises a capacitance sensor provided below the enlarged portion, and the manipulator receives the first operation as a change in capacitance due to contact of an operator with the capacitance sensor.

11. The X-ray diagnostic apparatus of claim 4, wherein the manipulator further comprises a pressure sensor provided below the enlarged portion, and the manipulator receives the first operation as a change in pressure due to contact of an operator with the pressure sensor.

12. The X-ray diagnostic apparatus of claim 4, wherein the enlarged portion of the manipulator has a planar shape comprising one or a plurality of recesses,
one or a plurality of first receivers configured to receive an operation from below with respect to the manipulator, wherein the one or the plurality of first receivers are provided below the one or the plurality of recesses, respectively, and
the manipulator receives the first operation by moving at least one of the one or the plurality of first receivers upward.

13. The X-ray diagnostic apparatus of claim 1, wherein the manipulator is immovably fixed to an operation panel in response to an operation of the manipulator from above.

14. The X-ray diagnostic apparatus of claim 13, wherein the manipulator comprises a support column extending upward from the operation panel, an enlarged portion provided on the support column and enlarged laterally from the support column, and a sliding portion provided slidably on the support column, and the manipulator receives the first operation when the sliding portion is moved upward.

15. The X-ray diagnostic apparatus of claim 1, further comprising:
a driver configured to drive movement of the top plate in a horizontal direction; and
processing circuitry configured to control the driver based on an instruction from an operator;
wherein when the releaser releases the movement restriction, the top plate can be controlled to move in the horizontal direction by the processing circuitry.

16. The X-ray diagnostic apparatus of claim 1, further comprising:
an operating force sensor configured to detect an operating force in the horizontal direction applied to the manipulator; and
processing circuitry configured to support movement of the top plate in the horizontal direction in accordance with the operating force in the horizontal direction detected by the operating force sensor;
wherein when the releaser releases the movement restriction, the top plate can be manually moved in the horizontal direction, and the processing circuitry supports the movement of the top plate in the horizontal direction.

17. The X-ray diagnostic apparatus of claim 1, further comprising processing circuitry configured to detect a user based on optical information acquired using an optical camera;
wherein when the manipulator receives the first operation and the processing circuitry detects that the user exists in front of the manipulator, the releaser releases the movement restriction.

18. The X-ray diagnostic apparatus of claim 17, wherein the optical camera is an optical camera provided on a ceiling of an inspection room, an optical camera provided on a side wall of the inspection room, or an optical camera attachable to a part of a body of the user.

19. The X-ray diagnostic apparatus of claim 1, further comprising processing circuitry configured to recognize a voice command from a user based on voice information input using a voice inputter;
wherein when the manipulator receives the first operation and the processing circuitry recognizes a voice command for releasing the movement restriction, the releaser releases the movement restriction.

20. A medical bed device, comprising:
a restrictor configured to set a movement restriction of a top plate in the horizontal direction, wherein a subject is placed on the top plate;
a manipulator configured to receive a releasing operation for releasing the movement restriction; and
a releaser configured to release the movement restriction when the manipulator receives a first operation that is the releasing operation from below.
